(19) United States
(12) Patent Application Publication
(10) Pub. No.: US 2024/0401046 A1
(43) Pub. Date: Dec. 5, 2024

(54) PRODUCTS AND METHODS FOR MYELIN PROTEIN ZERO SILENCING AND TREATING CMT1B DISEASE

(71) Applicant: RESEARCH INSTITUTE AT NATIONWIDE CHILDREN' S HOSPITAL, Columbus, OH (US)

(72) Inventors: Scott Quenton Harper, Powell, OH (US); Afrooz Rashnonejad, Columbus, OH (US)

(21) Appl. No.: 18/698,253

(22) PCT Filed: Oct. 7, 2022
(86) PCT No.: PCT/US22/77726

§ 371 Date: Apr. 3, 2024

Related U.S. Application Data
(60) Provisional application No. 63/253,357, filed on Oct. 7, 2021.

(51) Int. Cl. C12N 15/113; C12N 15/86

(52) U.S. Cl. CPC C12N 15/113; C12N 15/86; C12N 2310/141; C12N 2310/531; C12N 2320/31; C12N 2750/14143; C12N 2830/008

(57) ABSTRACT

RNA interference-based products and methods for inhibiting the expression of a mutant myelin protein zero (MPZ) gene in a cell or in the cells of a subject are disclosed. The disclosure includes microRNA that specifically target various regions of the MPZ gene to knock down expression of the aberrant protein. Additionally, the disclosure includes delivery of a nucleic acid encoding normal, wild-type, or functionally active MPZ protein. Additionally, the disclosure includes recombinant adeno-associated viruses to deliver nucleic acids encoding the microRNAs to knock down the expression of aberrant MPZ protein and/or to deliver nucleic acids encoding normal, wild-type, or functionally active MPZ protein. The disclosure includes methods of using these nucleic acids in the treatment of diseases associated with MPZ gene mutations including, but not limited to, Charcot-Marie-Tooth disease type 1B (CMT 1B) disease.

### INCORPORATION BY REFERENCE OF THE SEQUENCE LISTING

This application contains, as a separate part of disclosure, a Sequence Listing in computer-readable form (filename: 57017_Seqlisting.XML; Size: 111,501 bytes; Created: Oct. 3, 2022) which is incorporated by reference herein in its entirety.

### FIELD

Products and methods are provided for silencing or inhibiting the expression of the myelin protein zero (MPZ) gene (1q22), in which mutations of the gene are associated with underexpression of the wild-type MPZ protein and severe early-onset types of congenital hypomyelination, including various types of Charcot-Marie-Tooth (CMT) disease. MPZ is well known as a CMT-causative gene with wide phenotypic spectrum. Some mutations in MPZ gene cause many different types of hypomyelination diseases and CMT disease including, but not limited to, dominant intermediate CMT (DI-CMT), CMT type 1B (CMT1B), CMT type 2I (CMT2I), and CMT type 2J (CMT2J), and Dejerine-Sottas Syndrome (DSS or CMT type 3) disease. CMT1B disease presents with the manifestations of peripheral neuropathy including, but not limited to, distal muscle weakness and atrophy, foot deformities, and sensory loss. The disclosure provides a gene therapy approach for treating CMT1B using a nucleic acid encoding an artificial microRNA (miRNA) which specifically hybridizes to a target nucleic acid sequence encoding the MPZ gene, wherein binding of the complex to the target nucleic acid sequence results in knockdown of MPZ gene expression, along with using a nucleic acid encoding a codon-optimized MPZ gene (coMPZ or resMPZ) that is resistant to the artificial miRNA designed to knock down the MPZ gene. Delivery vehicles, such as recombinant adeno-associated viruses, deliver nucleic acids encoding the one or more MPZ microRNAs, as well as the coPMZ gene. These products and methods have application in the treatment of CMT1B disease and other disorders where aberrant MPZ expression is indicated.

### BACKGROUND

Charcot-Marie-Tooth disease (CMT) is a clinically and genetically heterogeneous collection of inherited peripheral neuropathies with a prevalence of up to 1 in 2,500. The CMT neuropathy type 1B (CMT1B) is the third-most common subtype of CMT1, accounting for 10 percent of cases affecting approximately 1 in 30,000 individuals. The hallmarks of CMT1B include slowly progressive distal muscle weakness and atrophy, foot drop and deformities, sensory loss, and absent reflexes with two typical onsets: very severe with early infantile and adolescence onset. CMT1B is caused by more than 200 different mutations in the Myelin Protein Zero (MPZ or P0) gene. The MPZ gene encodes the MPZ protein, the major protein in the myelin sheath. MPZ is an essential protein in maintaining a healthy and efficient peripheral nervous system. Accumulation of defect protein in Schwann cells causes demyelination and cell death overtime. The pathological mechanisms of CMT1B disease can be mostly divided into two major groups: (1) toxic gain-of-function mutations that directly impact normal myelination, and (2) defective unfolded protein response (UPR) or endoplasmic reticulum (ER) stress responses. Both mechanisms of disease will finally lead to the accumulation of mutant myelin protein in Schwann Cells (SCs), reduced myelination, muscle weakness and atrophy, and loss of sensation in the lower legs and feet. For example, the R98C mutation causes an early onset, severe disease due to retaining mutated MPZ protein in ER and defective UPR. Patients carrying this mutation have very low or almost no myelin. These patients are classified as CMT1B but also as Dèjèrine-Sottas-Syndrome (DSS), another defining of very severe CMT1B or congenital hypomyelination. There is no cure for this disease yet.

There remains a need in the art for products and methods of treatment for MPZ gene disorders including, but not limited to, CMT1B disease.

### SUMMARY

Provided herein are products, methods, and uses for treating mutations in the myelin protein zero (MPZ) gene and treating, ameliorating, delaying the progression of, and/or preventing diseases resulting from mutations in the MPZ gene.

The disclosure provides a nucleic acid comprising
a) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
b) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
c) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42;
d) a polynucleotide sequence that specifically hybridizes to a target nucleotide sequence on the MPZ gene, wherein the target nucleotide sequence is set forth in any one of SEQ ID NOs: 43-54; or
e) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

The disclosure provides a nucleic acid comprising
a) i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18; or
   ii) a polynucleotide sequence that specifically hybridizes to a target nucleotide sequence on the MPZ gene, wherein the target nucleotide sequence is set forth in any one of SEQ ID NOs: 43-54; and
b) i) a polynucleotide sequence encoding a human MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 1;
   ii) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 3; or
   iii) a polynucleotide sequence encoding an MPZ polypeptide sequence comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 4.

In some aspects, the nucleic acid further comprises a promoter or multiple promoters.

In some aspects, the promoter is a U6 promoter, a U7 promoter, an H19 promoter, a neuron-specific promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the actin promoter, the myosin promoter, the elongation factor-1a promoter, the hemoglobin promoter, the creatine kinase promoter, a Schwann cell-specific promoter, a myelin-specific promoter, or a native promoter. In some aspects, the promoter may be an MPZ promoter, a mini-MPZ promoter, a non-compact myelin associated protein (NCMPA or MP11) promoter, a PMP22 promoter, an MBP promoter, a SOX10 promoter, or a GAP43 promoter. In some aspects, the promoter used with the miMPZ is a U6 promoter. In some aspects, the promoter used with the MPZ replacement gene is an MPZ promoter or a mini-MPZ promoter. In some more specific aspects, the promoter is a U6 promoter, a U7 promoter, an H19 promoter, a neuron-specific promoter, or a Schwann cell-specific promoter. In some aspects, such Schwann cell-specific promoter is an MPZ promoter. In some aspects, the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5.

The disclosure provides a nanoparticle, extracellular vesicle, exosome, or vector comprising any of the nucleic acids of the disclosure or a combination of any one or more thereof.

In some aspects, the vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV), a self-complementary recombinant AAV (scAAV), or a single-stranded recombinant AAV (ssAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F. In some specific aspects, the AAV is AAV9, AAVrh.10, AAV-PHP.eB, AAVv66, or AAV-F.

The disclosure provides a composition comprising any of the nucleic acids of the disclosure or a combination of any one or more thereof. The disclosure provides a composition comprising any of the nanoparticles, extracellular vesicles, exosomes, vectors, or viral vectors of the disclosure, or a combination of any one or more thereof. In some aspects, the composition also comprises a pharmaceutically acceptable carrier.

The disclosure provides a method of reducing the expression of a mutant myelin protein zero (MPZ) gene in a cell comprising contacting the cell with any of the nucleic acids of the disclosure or a combination of any one or more thereof. In some aspects, the method comprises contact the cell with a nucleic acid comprising
a) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
b) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
c) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42;
d) a polynucleotide sequence that specifically hybridizes to a target nucleotide sequence on the MPZ gene, wherein the target nucleotide sequence is set forth in any one of SEQ ID NOs: 43-54; and/or
e) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

In some aspects, the method comprises contacting the cell with a nucleic acid comprising
a) i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18; or
   ii) a polynucleotide sequence that specifically hybridizes to a target nucleotide sequence on the MPZ gene, wherein the target nucleotide sequence is set forth in any one of SEQ ID NOs: 43-54; and
b) i) a polynucleotide sequence encoding a human MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 1;
   ii) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 3; and/or
   iii) a polynucleotide sequence encoding an MPZ polypeptide sequence comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 4.

In some aspects, the nucleic acid further comprises a promoter or multiple promoters. In some aspects, the promoter is a U6 promoter, a U7 promoter, an H19 promoter, a neuron-specific promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the actin promoter, the myosin promoter, the elongation factor-1a promoter, the hemoglobin promoter, the creatine kinase promoter, a Schwann cell-specific promoter, a myelin-specific promoter, or a native promoter. In some aspects, the promoter may be an MPZ promoter, a non-compact myelin associated protein (NCMPA or MP11) promoter, a PMP22 promoter, an MBP promoter, a SOX10 promoter, or a GAP43 promoter. In some aspects, the promoter used with the miMPZ is a U6 promoter. In some aspects, the promoter used with the MPZ replacement gene is an MPZ promoter or a mini-MPZ promoter. In some more specific aspects, the promoter is a U6 promoter, a U7 promoter, an H19 promoter, a neuron-specific promoter, or a Schwann cell-specific promoter. In some aspects, such Schwann cell-specific promoter is an MPZ promoter or a mini-MPZ promoter. In some aspects, the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5. In some aspects, the nucleic acid is in a nanoparticle, extracellular vesicle, exosome, or vector. In some aspects, the nucleic acid is in a viral vector. In some aspects, the nucleic acid, nanoparticle, extracellular vesicle, exosome, or vector is in a composition. In some aspects, the cell is a neuronal cell. In some aspects, the neuronal cell is a Schwann cell. In some aspects, the cell is a human cell. In some aspects, the cell is in a human subject.

The disclosure provides a method of treating a subject comprising a mutant myelin protein zero (MPZ) gene, the method comprising administering to the subject an effective amount of any of the nucleic acids of the disclosure or a combination of any one or more thereof. In some aspects, the method comprises administering to the subject an effective amount of a nucleic acid comprising
a) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
b) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
c) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42;
d) a polynucleotide sequence that specifically hybridizes to a target nucleotide sequence on the MPZ gene, wherein the target nucleotide sequence is set forth in any one of SEQ ID NOs: 43-54; and/or
e) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

In some aspects, the method comprises administering to the subject an effective amount of a nucleic acid comprising
a) i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18; or
   ii) a polynucleotide sequence that specifically hybridizes to a target nucleotide sequence on the MPZ gene, wherein the target nucleotide sequence is set forth in any one of SEQ ID NOs: 43-54; and
b) i) a polynucleotide sequence encoding a human MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 1;
   ii) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 3 or 6; or
   iii) a polynucleotide sequence encoding an MPZ polypeptide sequence comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 4.

In some aspects, the nucleic acid further comprises a promoter or multiple promoters. In some aspects, the promoter is a U6 promoter, a U7 promoter, an H19 promoter, a neuron-specific promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the actin promoter, the myosin promoter, the elongation factor-1a promoter, the hemoglobin promoter, the creatine kinase promoter, a Schwann cell-specific promoter, a myelin-specific promoter, or a native promoter. In some aspects, the promoter may be an MPZ promoter, a non-compact myelin associated protein (NCMPA or MP11) promoter, a PMP22 promoter, an MBP promoter, a SOX10 promoter, or a GAP43 promoter. In some aspects, the promoter used with the miMPZ is a U6 promoter. In some aspects, the promoter used with the MPZ replacement gene is an MPZ promoter or a mini-MPZ promoter. In some more specific aspects, the promoter is a U6 promoter, a U7 promoter, an H19 promoter, a neuron-specific promoter, or a Schwann cell-specific promoter. In some aspects, such Schwann cell-specific promoter is an MPZ promoter or a mini-MPZ promoter. In some aspects, the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5. In some aspects, the nucleic acid is present in a nanoparticle, extracellular vesicle, exosome, or vector. In some aspects, the nucleic acid is in a viral vector. In some aspects, the viral vector is AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F. In some specific aspects, the AAV is AAV9, AAVrh.10, AAV-PHP.eB, AAVv66, or AAV-F. In some aspects, the nucleic acid, nanoparticle, extracellular vesicle, exosome, or vector is in a composition. In some aspects, the subject is a human subject. In some aspects, the subject suffers from a hypomyelination disease or a Charcot-Marie-Tooth disease. In some aspects, the disease is CMT (DI-CMT), CMT type 1B (CMT1B), CMT type 2I (CMT2I), CMT type 2J (CMT2J), or Dejerine-Sottas Syndrome (DSS or CMT type 3) disease.

The disclosure provides a method of reducing expression of a mutant myelin protein zero (MPZ) gene in a cell and expressing a functional MPZ protein in a cell, the method comprising delivering to the cell an effective amount of
(a) a nucleic acid comprising one or more of
   (i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
   (ii) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
   (iii) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42; and/or
   (iv) a polynucleotide sequence that specifically hybridizes to a target nucleotide sequence on the MPZ gene, wherein the target nucleotide sequence is set forth in any one of SEQ ID NOs: 43-54; and
(b) a nucleic acid comprising a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

In some aspects, any one or more of the nucleic acids further comprises a promoter or multiple promoters. In some aspects, the promoter is a U6 promoter, a U7 promoter, an H19 promoter, a neuron-specific promoter, or a Schwann cell-specific promoter. In some aspects, the Schwann cell-specific promoter is an MPZ promoter or a mini-MPZ promoter. In some aspects, the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5. In some aspects, the cell is a neuronal cell. In some aspects, the neuronal cell is a Schwann cell. In some aspects, the cell is a human cell. In some aspects, the cell is in a human subject. In some aspects, the subject suffers from a hypomyelination disease or a Charcot-Marie-Tooth disease. In some aspects, the disease is CMT (DI-CMT), CMT type 1B (CMT1B), CMT type 2I (CMT2I), CMT type 2J (CMT2J), or Dejerine-Sottas Syndrome (DSS or CMT type 3) disease. In some aspects, the nucleic acid is delivered to the cell in a nanoparticle, an extracellular vesicle, an exosome, or a vector, or a combination of any one or more thereof. In some aspects, the vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV), a self-complementary recombinant AAV (scAAV), or a single-stranded recombinant AAV (ssAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, or AAVv66, or AAV-F. In some specific aspects, the AAV is AAV9, AAVrh.10, AAV-PHP.eB, AAVv66, or AAV-F. In some aspects, the nucleic acid comprising the polynucleotide sequence comprising or encoding the MPZ miRNA and/or the polynucleotide sequence that specifically hybridizes to a target nucleotide sequence on the MPZ gene, and the nucleic acid comprising the polynucleotide sequence encoding the codon-optimized MPZ DNA are delivered to the cell at the same time. In some aspects, the nucleic acids are delivered to the cell in the same vector. In some aspects, the nucleic acid comprising the polynucleotide sequence comprising or encoding the MPZ miRNA and/or the polynucleotide sequence that specifically hybridizes to a target nucleotide sequence on the MPZ gene, and the nucleic acid comprising the polynucleotide sequence encoding the codon-optimized MPZ DNA are delivered to the cell at different times.

The disclosure provides a method of treating a subject suffering from aberrant expression of a mutant myelin protein zero (MPZ) gene, the method comprising reducing expression of the mutant MPZ gene and expressing functional MPZ protein in the subject, the method comprising delivering to the subject an effective amount of
(a) a nucleic acid comprising one or more of
   (i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
   (ii) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
   (iii) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42; and/or
   (iv) a polynucleotide sequence that specifically hybridizes to a target nucleotide sequence on the MPZ gene, wherein the target nucleotide sequence is set forth in any one of SEQ ID NOs: 43-54; and
(b) a nucleic acid comprising a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

The disclosure also provides a method of treating a subject suffering from aberrant expression of a mutant myelin protein zero (MPZ) gene, the method comprising expressing functional MPZ protein in the subject by delivering to the subject an effective amount of a nucleic acid comprising a polynucleotide sequence encoding an MPZ DNA or a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1, 3, and 6.

In some aspects, any one or more of the nucleic acids further comprises a promoter or multiple promoters. In some aspects, the promoter is a U6 promoter, a U7 promoter, an H19 promoter, a neuron-specific promoter, or a Schwann cell-specific promoter. In some aspects, the Schwann cell-specific promoter is an MPZ promoter or a mini-MPZ promoter. In some aspects, the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5. In some aspects, the subject is a human. In some aspects, the subject suffers from a hypomyelination disease or a Charcot-Marie-Tooth disease. In some aspects, the disease is CMT (DI-CMT), CMT type 1B (CMT1B), CMT type 2I (CMT2I), CMT type 2J (CMT2J), or Dejerine-Sottas Syndrome (DSS or CMT type 3) disease. In some aspects, the nucleic acid is delivered to the subject in a nanoparticle, an extracellular vesicle, an exosome, or a vector, or in a combination of any one or more thereof. In some aspects, the vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV), a self-complementary recombinant AAV (scAAV), or a single-stranded recombinant AAV (ssAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, or AAVv66, or AAV-F. In some specific aspects, the AAV is AAV9, AAVrh.10, AAV-PHP.eB, AAVv66, or AAV-F. In some aspects, the nucleic acid comprising the polynucleotide sequence comprising or encoding the MPZ miRNA and/or the polynucleotide sequence that specifically hybridizes to a target nucleotide sequence on the MPZ gene, and the nucleic acid comprising the polynucleotide sequence encoding the codon-optimized MPZ DNA are delivered to the subject at the same time. In some aspects, the nucleic acids are delivered to the subject in the same vector. In some aspects, the nucleic acid comprising the polynucleotide sequence comprising or encoding the MPZ miRNA and/or the polynucleotide sequence that specifically hybridizes to a target nucleotide sequence on the MPZ gene, and the nucleic acid comprising the polynucleotide sequence encoding the codon-optimized MPZ DNA are delivered to the cell at different times.

The disclosure provides use of any of the nucleic acids of the disclosure or a combination thereof. The disclosure provides use of any of the nanoparticles, extracellular vesicles, exosomes, or vectors of the disclosure. The disclosure provides the use of any of the viral vectors of the disclosure. The disclosure provides the use of any of the compositions of the disclosure. In some aspects, the use is for the preparation of a medicament for reducing expression of a mutant myelin protein zero (MPZ) gene in a cell. In some aspects, the cell is in a human subject. Thus, in some other aspects, the cell is ex vivo or in vitro and outside a subject. In some aspects the use is for treating a subject comprising a mutant myelin protein zero (MPZ) gene. In some aspects, the subject is a human subject. In some aspects, the subject suffers from a hypomyelination disease or a Charcot-Marie-Tooth disease. In some aspects, the disease is CMT (DI-CMT), CMT type 1B (CMT1B), CMT type 2I (CMT2I), CMT type 2J (CMT2J), or Dejerine-Sottas Syndrome (DSS or CMT type 3) disease.

The disclosure provides further that any of the nucleic acids, nanoparticles, extracellular vesicles, exosomes, vectors, viral vectors, compositions, or medicaments described herein, in some aspects, is formulated for intramuscular injection, oral administration, subcutaneous, intradermal, or transdermal transport, injection into the blood stream, or for aerosol administration.

Other features and advantages of the disclosure will become apparent from the following description of the drawings and the detailed description. It should be understood, however, that the drawings, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

FIG. 1 provides a schematic of human and mouse MPZ cDNA. The most prevalent dominant mutations or small deletions in the MPZ cDNA causing CMT1B are indicated. Stars represent conserved areas on the mouse and human MPZ sequence. Bold horizontal lines indicate the targeting position for designed miRNAs on MPZ cDNA. The miRNAs were designed to target highly conserved sequences to target both human and mouse MPZ sequences in preclinical studies in mice using one single vector.

FIGS. 2A-L show each of the MPZ microRNA (miMPZ) nucleotide sequences of the disclosure, i.e., FIG. 2A shows miMPZ_225; FIG. 2B shows miMPZ_226; FIG. 2C shows miMPZ_315; FIG. 2D shows miMPZ_316; FIG. 2E shows miMPZ_317; FIG. 2F shows miMPZ_718; FIG. 2G shows miMPZ_719; FIG. 2H shows miMPZ_720; FIG. 2I shows miMPZ_721; FIG. 2J shows miMPZ_722; FIG. 2K shows miMPZ_723; and FIG. 2L shows miMPZ_1852. In each of FIGS. 2A-L, the top sequences indicate the DNA templates synthesis by PCR using primer pairs explained in the text. Each DNA template transcribes a respective miMPZ. The folded miRNA transcripts are shown as hairpin structures. The mature miMPZ sequences arise following processing in target cells by host miRNA processing machinery (including Drosha, DGCR8, Dicer, and Exportin-5). Sequences shaded in gray indicate restriction sites used for cloning each miRNA into the U6T6 vector. CTCGAG (SEQ ID NO: 79) is an XhoI site and ACTAGT (SEQ ID NO: 80) is a SpeI site (CUCGAG (SEQ ID NO: 81) and ACUAGU (SEQ ID NO: 82) in RNA, where the U is a uracil base). The underlined, italicized sequence indicates the mature miRNA antisense guide strand that ultimately helps catalyze cleavage of the DUX4 target mRNA. This sequence is also underlined in the miRNA hairpin portions of this diagram. The arrowheads indicate Drosha- and Dicer-catalyzed cleavage sites, respectively. The numbers 13, 35, 53, and 75 are provided for orientation. The sequences between (and including) positions 35-53 are derived from the natural human mir-30a sequence, except the A at position 39, which is a G is the normal mir-30a sequence. This nucleotide was changed to an A to facilitate folding of the miRNA loop, based on in silico RNA folding models. The base of the stem (5′ of position 13 and 3′ of position 75) was derived from the mir-30a structure and sequence with some modifications, depending on the primary sequence of the guide strand. Specifically, the nucleotide at position 13 can vary to help facilitate a required mismatched between the position 13 and 75 nucleotides. This bulged structure is hypothesized to facilitate proper Drosha cleavage.

FIGS. 3A-D show results of preliminary screening miMPZ of the disclosure for efficacy in knocking down MPZ expression. FIG. 3A shows a schematic diagram of dual luciferase plasmid used in various tests described in the disclosure. To generate a dual luciferase reporter plasmid, the human MPZ cDNA was cloned downstream of the Renilla luciferase gene as 3′ untranslated region (3′UTR) in psiCheck2 plasmid (Promega). This conformation does not produce a Luciferase—MPZ fusion protein, since the MPZ sequences are placed after the Renilla luciferase stop codon. Instead, a fusion mRNA is produced, in which the MPZ sequences act as 3′ UTR of Renilla luciferase. As a result, any effective MPZ-targeted miRNA will reduce the Renilla Luciferase-MPZ fusion mRNA, which subsequently decreases Renilla luciferase protein expression in transfected cells. There is a separate Firefly luciferase gene expressed under a different promoter in same plasmid, which does not contain any MPZ sequences and is therefore unaffected by miMPZs. FIG. 3B shows luciferase assay results of initial miMPZ efficacy screens. All samples in this assay were normalized to cells co-transfected with the reporter vector and the U6T6 plasmid. All miMPZs, except miMPZ_720, efficiently reduced Renilla Luciferase expression. miMPZ 225, 226, 721, and 723 demonstrated the greatest efficiency in this experiment. FIG. 3C shows a Western blot indicating that miMPZ 225, 226, 317, 721, 723 reduced MPZ expression significantly in co-transfected HEK293 cells. FIG. 3D shows the relative expression of hMPZ related to untreated controls with qRT-PCR demonstrating that miMPZ reduces MPZ gene expression. Based on these results, miMPZ_225, 226, 317, 721, and 723 reduced MPZ mRNA levels most significantly in co-transfected HEK293 cells. These miMPZ indicate, in various aspects, that they are efficient to target and reduce MPZ R98C expression level in co-transfected HEK293 cells.

FIGS. 4A-C shows reduction of MPZ R98C expression achieved by the artificial miMPZs described herein. FIG. 4A demonstrates the luciferase assay results of miMPZ efficacy screens on targeting mutant MPZ R98C in HEK293 cells. All samples in this assay were normalized to cells co-transfected with reporter vector and the U6T6 plasmid. All miMPZs efficiently reduced Renilla Luciferase expression. FIG. 4B demonstrates the Western blot results of MPZ R98C knockdown by various miMPZs in co-transfected HEK293 cells. miMPZ 225, 226, 317, 719, 721, and 723 reduced R98C expression more significantly in co-transfected HEK293 cells. FIG. 4C demonstrates the qRT-PCR results of MPZ R98C expression level in HEK293 cells co-transfected with each miMPZ and R98C expression plasmids. All MPZ miRNAs reduced R98C mRNA level significantly compared to untreated R98C in co-transfected cells.

FIG. 5 shows SEQ ID NO: 6, the sequence of an MPZ expression cassette made of 1200 bp of human MPZ promoter (ENSG00000158887), 63 bp human MPZ 5′UTR, and 747 bp partially codon optimized MPZ cDNA. It also has SV40 polyA signal and CMV amplicon sequences. The CMV amplicon is used for AAV titration using qRT-PCR or ddPCR assays. The miMPZs are cloned into the NsiI or NotI sites located after the CMV amplicon. In some exemplary aspects, the expression cassette is cloned into an AAV pre-plasmid using two XbaI restriction enzyme sites at each end. The order of sequences in the coMPZ expression cassette is as follows: XBAI-stui-SaIl-kasi-human MPZ promoter (1200 bp)-NheI-human MPZ 5′UTR- partially codon optimized human mpz cds-SPEI-ndei-SV40 PA-paci-CMV AMPLICON FOR DRP-NSII-noti-XBAI. Various MPZ promoters including human, rat, mouse or incombination with universal enhancers such as CMV or Schwann Specific enhancers such as PMP22 also can be used in this construct.

FIGS. 6A-C show the base-paring between mi225 and human and mouse MPZ, and the resistance of coMPZ to miMPZ knockdown. FIG. 6A shows miMPZ-225 perfect base-pairing with wild-type human and mouse MPZ. FIG. 6B shows mismatches between miMPZ225 and coMPZ. Mutated nucleotides (bold) are in wobble positions and maintain wild-type MPZ amino acids. The G-U is a wobble base pair. FIG. 6C shows qRT-PCR results. miMPZ-225 reduced wild-type human MPZ by >75% in co-transfected HEK293 cells, while coMPZ was comparatively resistant to high levels of silencing. The slight decrease in coMPZ expression in this experiment might reflect differences in transcription or qRT-PCR detection of the two MPZ transcripts.

### DETAILED DESCRIPTION

The disclosure provides a novel strategy to accomplish the inhibition of mutant Myelin Protein Zero (MPZ or P0) gene expression post-transcriptionally by repressing or inhibiting mutant MPZ gene expression and protein production because the expression of mutant MPZ protein is known to cause congenital neuropathy with hypomyelination including, but not limited to, Charcot-Marie-Tooth disease (CMT). Thus, in some aspects, the products, methods, and uses described herein are used in treating, ameliorating, delaying the progression of, and/or preventing neuropathy with hypomyelination including, but not limited to, CMT.

CMT is a clinically and genetically heterogeneous collection of inherited peripheral neuropathies with a prevalence of up to 1 in 2,500. Charcot-Marie-Tooth type 1B (CMT1B) is the third most common form of inherited demyelinating neuropathy, accounting for 10 percent of cases. CMT1B is caused by DNA mutations, i.e., autosomal dominant gain-of-function mutations in the MPZ gene. MPZ is an essential protein, the major protein in the myelin sheath required for maintaining a healthy and efficient peripheral nervous system. Accumulation of defective MPZ protein in Schwann cells, which are supporting cells in peripheral nerves, causes progressive nerve damage, leading to CMT1B symptoms. The hallmarks of CMT1B include slowly progressive distal muscle weakness and atrophy, foot drop and deformities, sensory loss, and absent reflexes with two typical onsets: very severe with early infantile and adolescence onset. Treatment of Dèjèrine-Sottas-Syndrome (DSS), another defining of very severe CMT1B or congenital hypomyelination, is included in the methods and uses of the disclosure.

MPZ is a 27-kDa single membrane glycoprotein (Magnaghi et al., Brain Research Reviews, 2011, 37 (1-3): 360-371) expressed by myelinating Schwann cells. It accounts for over 50% of all proteins in the peripheral nervous system, making it the most common protein expressed in the PNS (Shy, Journal of the Neurological Sciences, 2006, 242 (1-2): 55-66). The vast majority of MPZ mutations causing CMT1B are dominantly inherited. Various pathological mechanisms are suggested for each MPZ gene mutation. However, the main pathological mechanisms of dominant mutations in MPZ gene can be divided into two major groups: (1) toxic gain-of-function mutations that directly impact normal myelination, and (2) defective unfolded protein response (UPR) or endoplasmic reticulum (ER) stress responses, which induce apoptosis (Bai et al., Ann Clin Transl Neurol, 2018, 5:445-455; Bai et al., Rare Dis, 2013, 1:e24049; Wrabetz et al., J Neurosci, 2006, 26:2358-2368; Fratta et al., Hum Mol Genet, 2019, 1; 28(1):124-132). Intracellular accumulation of mutant protein is common to both mechanisms. For example, R98C mutation causes an early onset, severe disease due to retaining mutant MPZ protein in the ER and defective UPR. Patients carrying this mutation have very low or almost no myelin. Some patients may never walk or solely use wheelchairs by the end of their first decade. The median motor nerve conduction velocities (MNCVs) are generally less than 6 m/see while normal is around 40 m/sec. These patients are classified as CMT1B.

Additional diseases associated with aberrant MPZ expression include, but are not limited to, severe early-onset types of congenital hypomyelination, Dejerine-Sottas Syndrome or disease (DSS or CMT type 3). Some mutations in the MPZ gene also cause dominant intermediate Charcot-Marie-Tooth disease (DI-CMT), CMT type 2I (CMT2I), and CMT type 2J (CMT2J).

MPZ levels must exist within a narrowly defined range. Lack of Mpz expression in homozygous Mpz null mice causes poorly compacted myelin sheaths (Grandis et al., Hum Mol Genet, 2008, 1; 17(13):1877-89), dysregulation of myelin-specific gene expression, and abnormalities of myelin protein localization in Schwann cells (Bai et al., Ann Clin Transl Neurol, 2018, 5:445-455; Wrabetz et al., J Neurosci, 2006, 26(8): 2358-2368). The heterozygous Mpz null mice demonstrate a very mild phenotype similar to late, adult-onset forms of CMT1B. Interestingly, more than 80% (>1.8-fold) overexpression of the Mpz gene also inhibits Schwann cell association with axons and disrupts myelination (Bai et al., Rare Dis, 2013, 1:e24049).

Several transgenic CMT1B mouse models have been created and have confirmed that dominant mutations in the MPZ gene cause peripheral neuropathy (Saporta et al., Brain, 2012, 135(7):2032-47; Wrabetz et al., supra; Giese et al., Cell, 1992, 71:565-576). One of the CMT1B mouse models is the MPZR98C transgenic line. The R98C ‘knock-in’ mouse model of CMT1B (Saporta et al. (supra); provided by Prof. Michael Shy, University of Iowa) was generated by site-directed mutagenesis using homologous recombination method. MPZR98C arrests Schwann cell development in a mouse model of early-onset CMT1B. These mice display histological and behavioral phenotypes. Both heterozygous (R98C/+) and homozygous (R98C/R98C) mice develop weakness, abnormal nerve conduction velocities, and morphologically abnormal myelin; R98C/R98C mice are more severely affected. These mice demonstrate accumulation of MpzR98C in the endoplasmic reticulum and a developmental delay in myelination similar to that in patients harboring the same mutation.

The disclosure includes the use of homozygous R98C/R98C and heterozygous R98C/+ mice in the gene therapy studies. Homozygous mice have a slow, unsteady gait that is apparent by the time of weaning at ˜21 days after birth with persistent tremors during walking. Heterozygous R98C/+ mice show no consistent, observable clinical abnormalities up to at least 1 year of age. However, both heterozygous and homozygous mice demonstrate abnormalities on tests of motor performance. R98C/R98C mice cannot maintain their balance on the rotating rod, and R98C/+ mice performed significantly worse than wild-type mice. The homozygous and heterozygous mice have slowed motor nerve conduction velocity (MNCV) of ˜4 m/s and ˜15 m/s, respectively, compared with MNCV of ˜40 m/s in wild-type mice at 6-8 weeks of age (MpzR98C arrests Schwann cell development in a mouse model of early-onset Charcot-Marie-Tooth disease type 1B) (Saporta et al., supra). Therefore, the homozygous mice are more relevant to severe, early-onset CMT1B (DSS), and heterozygous mice produce the late-onset form of CMT1B. Thus, in various aspects, the MPZR98C mice are used as a model. Thus, MpzR98C are used in various aspects of the disclosure as a treatment model (http-colon-forward slash-forward slash-www.medlink.com-forward slash-article-forward slash-charcot-marie-tooth-disease-type-1 b). Upon optimizing the therapeutic vectors and doses in these mice, additional mouse models are included for use in the disclosure to investigate the applicability of the proposed therapeutic strategy for various MPZ mutations.

Mouse models for milder forms of CMT1B, such as S63del (Wrabetz et al., supra) and H39P, two other common gain-of-function variants represent a childhood and an adult-onset CMT1B, respectively, and these models are included in various aspects of the disclosure. The S63del mice develop evident neuromuscular disorders between 4 and 8 weeks of age, characterized by tremor, ataxia, weakness, and muscle atrophy in the hindlimbs. S63del mice also develop noted hypomyelination with occasional nude axons and an onion bulb (concentrically layered Schwann-cell process surrounding nerve fibers) at the age of 6 months, increasing number after age 1 year old. The MNCV motor is reduced by 50% in S63del//+/− mice (Wrabetz et al., supra).

The disclosure provides a novel strategy to accomplish defective or mutant Myelin Protein Zero (MPZ or P0) gene silencing at the mRNA level using RNA inhibition (RNAi). The MPZ inhibitory RNAs include, but are not limited to, antisense RNAs, small inhibitory RNAs (siRNAs), short hairpin RNAs (shRNAs) or artificial microRNAs (MPZ miRNAs) that inhibit expression of the wild-type and mutant MPZ gene.

MPZ miRNAs can specifically bind to a segment of a messenger RNA (mRNA) encoded by a human MPZ gene (represented by SEQ ID NO: 1 which is a human MPZ cDNA), wherein the segment is conserved relative to mRNA encoded by the wild-type mouse MPZ gene (represented by SEQ ID NO: 82 which is a mouse MPZ cDNA). For example, a MPZ miRNA can specifically bind a mRNA segment that is complementary to a sequence within nucleotides 225-247, 315-318, or 718-744 of SEQ ID NO: 1.

The disclosure focuses on inhibiting mutated MPZ expression in Schwann cells while maintaining the healthy levels of the normal MPZ protein. To accomplish this, specifically designed artificial miRNAs to silence endogenous mutant and wild-type MPZ expression are provided with simultaneously replacing wild-type MPZ expression with a miRNA-resistant (rMPZ) gene. As used herein, the term “miRNA-resistant (rMPZ)” is used interchangeably with “resistant MPZ (resMPZ)” or “codon-optimized resistant MPZ (coMPZ)”. Thus, the disclosure provides miRNAs (called miMPZ) designed to specifically and equally target both human MPZ and mouse Mpz genes, with no predicted non-specific binding to other transcripts. This strategy allows the translation of results from murine models to human models, particularly important in clinical trials.

This disclosure provides products, methods, and uses for directly targeting the genetic cause underlying CMT disease, including CMT1B disease, and the MPZ gene itself. This disclosure provides a universal therapy applicable for all early and late-onset forms of CMT1B caused by dominant mutations in the MPZ gene. Using miRNA-mediated silencing of the MPZ gene is a novel approach to CMT1B treatment. miRNAs are highly conserved, small (approximately 22 nucleotides long), non-coding RNA molecules that negatively regulate the expression of many genes at the post-transcriptional level (He et al., Nat Rev Genet, 2004, 5:522-531; Carrington et al., Science, 2003, 301:336-338). Since natural miRNAs are implicated in several disease phenotypes and regulate more than one endogenous gene transcript, novel approaches utilize artificial miRNAs for therapeutic purposes (Hammond, Trends Mol Med, 2006, 12:99-101; McBride et al., Proc Natl Acad Sci USA, 2008, 105:5868-5873).

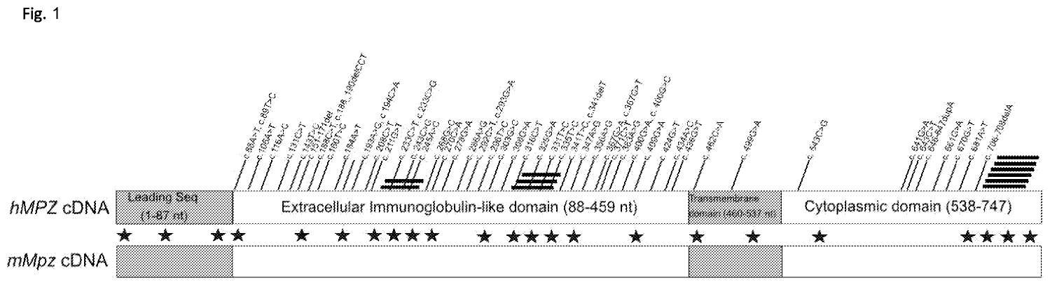

The disclosure provides engineered artificial miRNAs based on the natural human miR-30, by maintaining essential structural and sequence elements required for normal miRNA biogenesis but replacing the mature mir-30 sequences with those targeting the MPZ gene of interest. These new artificial miRNAs contain 22-nt of perfect complementarity with the MPZ gene. The miRNAs of the disclosure (miMPZ) are designed to target highly conserved sequences between the mouse and human MPZ cDNA. The miMPZ-6 to 11 are designed to a region of the MPZ gene which does not have any reported mutations associated with CMT1B (FIG. 1). FIG. 1 provides a schematic of human and mouse MPZ cDNA, showing where the most prevalent dominant mutations or small deletions causing CMT1B are indicated and the miRNAs of the disclosure were designed so that a single miRNA could target both human and mouse MPZ sequences.

In some aspects, the disclosure includes the treatment or amelioration of CMT1B by reducing defective MPZ protein and replacing it with healthy MPZ protein. This strategy “knockdown and replace” is accomplished by using gene therapy to deliver a MPZ-reducing molecule called a microRNA (miRNA), along with a healthy copy of the MPZ gene, to Schwann cells. Thus, in some aspects, the products and methods described herein are used in the treatment of MPZ gene disorders including, but not limited to CMT diseases, such as CMT1B.

The disclosure provides data showing that several miMPZs significantly reduced human mutant and wildtype MPZ expression by more than 80% in MPZ-overexpressing HEK293 cells. The disclosure also provides data that demonstrate that rMPZ expression is resistant to miMPZ silencing in transiently transfected HEK293 cells.

The disclosure provides various nucleic acids and polypeptides. The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence. The disclosure includes various polypeptides comprising, consisting essentially of, or consisting of the various amino acid sequences described herein. In some aspects, the polypeptide comprises the amino acid sequence. In some aspects, the polypeptide consists essentially of the amino acid sequence. In some aspects, the polypeptide consists of the amino acid sequence.

In some aspects, the nucleic acid comprises the nucleotide sequence encoding human MPZ set forth in the nucleotide sequence set forth in SEQ ID NO: 1. In various aspects, the nucleic acid is an isoform or variant of the nucleotide sequence encoding human MPZ comprising the nucleotide sequence set forth in SEQ ID NO: 1. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 1.

In some aspects, the polypeptide is a human MPZ polypeptide comprising the amino acid sequence set forth in SEQ ID NO: 4. In various aspects, the polypeptide is an isoform or variant of the human MPZ polypeptide comprising the amino acid sequence set forth in SEQ ID NO: 4. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the amino acid sequence set forth in SEQ ID NO: 4.

In some aspects, therefore, the nucleic acid comprises a nucleotide sequence encoding human MPZ set forth in the amino acid sequence set forth in SEQ ID NO: 4. In various aspects, the nucleic acid is an isoform or variant of a nucleotide sequence encoding human MPZ set forth in the amino acid sequence set forth in SEQ ID NO: 4. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to a nucleotide sequence encoding the polypeptide comprising the amino acid sequence set forth in SEQ ID NO: 4.

In some aspects, the nucleic acid comprises the nucleotide sequence encoding human MPZ 3′UTR set forth in SEQ ID NO: 2. The target site in the 3′UTR is optimized to make it resistant to silencing by the miRNA. In various aspects, the nucleic acid is an isoform or variant of the nucleotide sequence encoding human MPZ 3′UTR comprising the nucleotide sequence set forth in SEQ ID NO: 2. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 2.

RNA inhibition (RNAi) is described herein as an effective long-term treatment for dominant genetic disorders. As an example, products and methods are provided for treating a subject with an MPZ gene mutation by knocking down both wild-type and mutant forms of the involved gene(s), while also delivering an RNAi-resistant replacement MPZ gene. As an example, products and methods are described herein for knocking down the expression of a mutant MPZ gene and wild-type MPZ gene in a subject. The methods utilize MPZ inhibitory RNAs to knock down mutant MPZ gene expression. The methods also provide an RNAi-resistant replacement MPZ gene. Use of the methods and products is indicated, for example, in preventing, treating, or ameliorating diseases associated with a mutation in the MPZ gene, such as, for example, Charcot-Marie-Tooth disease type 1B (CMT1B) disease.

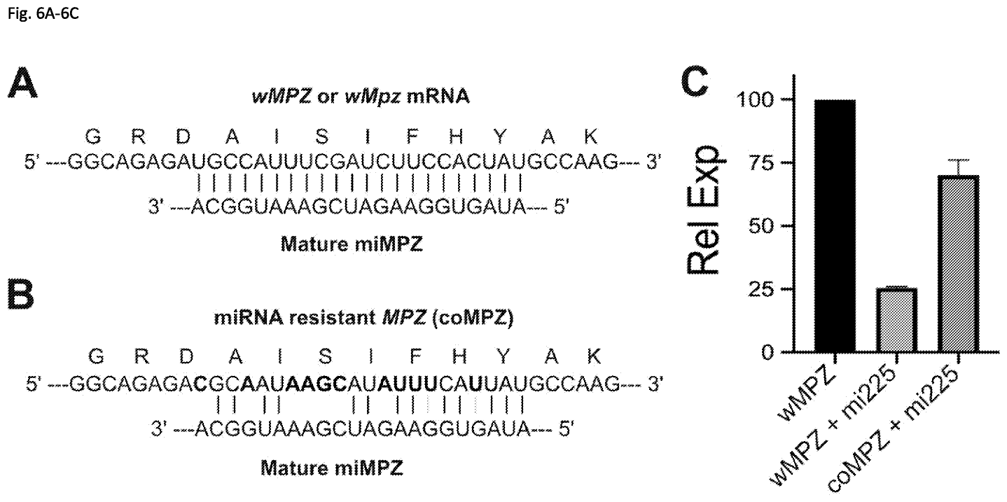

All miRNAs described herein were designed to target both wild-type and mutant MPZ genes. Because CMT1B is caused by autosomal dominant mutations, only one mutant allele is enough to cause CMT1B symptoms. Patients suffering from MPZ mutations can have one wild type and one mutant MPZ allele or two mutant MPZ alleles. Patients with only one mutant MPZ allele will have a milder phenotype compared to those with two mutant MPZ alleles. The miRNAs described herein were designed to target and silence both alleles. Additionally, because the expression of wild-type MPZ allele is necessary to maintain a healthy myelin sheath, an miRNA-resistant MPZ gene was designed by codon optimizing the MPZ cDNA sequence and mutating the miRNA target sites on the gene (as explained herein and as shown in FIG. 6A-B).

An exemplary miRNA-resistant MPZ gene is a codon-optimized MPZ cDNA (alternately referred to as a resistant MPZ (resMPZ or rMPZ) or coMPZ) sequence of the disclosure is set forth in SEQ ID NO: 3 or found within the MPZ expression cassette set out in SEQ ID NO: 6. The terms “miRNA-resMPZ”, “resMPZ”, “rMPZ”, “coMPZ”, as used herein, are interchangeable.

RNAi-resistant replacement MPZ genes are provided. An “RNAi-resistant replacement MPZ gene” has a nucleotide sequence the expression of which is not knocked down by the MPZ miRNAs described herein but the nucleotide sequence still encodes a MPZ protein that has MPZ protein activity. An exemplary RNAi-resistant replacement MPZ gene is set out in SEQ ID NO: 3 or found within the MPZ expression cassette set out in SEQ ID NO: 6.

Thus, the disclosure provides a nucleic acid for replacing mutant MPZ or wild-type MPZ which is down-regulated by MPZ microRNA (miRNA). In some aspects, the nucleic acid is codon-optimized to be resistant to exposure of MPZ miRNA. In some aspects, the nucleic acid comprises the nucleotide sequence encoding partially codon-optimized human MPZ (human coMPZ) set forth in SEQ ID NO: 3. In various aspects, the nucleic acid is an isoform or variant of the nucleotide sequence encoding partially codon-optimized human MPZ comprising the nucleotide sequence set forth in SEQ ID NO: 3. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 3.

In some aspects, the nucleic acid for replacing mutant MPZ or wild-type MPZ comprises the nucleotide sequence encoding a human MPZ promoter comprising the nucleotide sequence set forth in SEQ ID NO: 5. In various aspects, the nucleic acid is an isoform or variant of the nucleotide sequence encoding human MPZ promoter comprising the nucleotide sequence set forth in SEQ ID NO: 5. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 5.

In some aspects, the nucleic acid comprises a nucleotide sequence comprising a human MPZ expression cassette which comprises a human MPZ promoter, a human MPZ 5′UTR, and human partially codon-optimized MPZ cDNA. In some aspects, such nucleic acid comprises the nucleotide sequence set forth in SEQ ID NO: 6. In various aspects, the nucleic acid is an isoform or variant of the nucleotide sequence set forth in SEQ ID NO: 6. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 6.

Exemplary RNAi-resistant replacement MPZ genes are set out in any one of more of SEQ ID NOs: 3 and 6, or a variant thereof comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 3 and 6. Exemplary RNAi-resistant replacement MPZ genes further comprise an MPZ promoter comprising the nucleotide sequence set out in SEQ ID NO: 5, or a variant thereof comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5.

Table 1, set out below, provides various nucleotide and amino acid sequences of the disclosure. In particular, Table 1 provides sequences of human MPZ cDNA, 3′UTR DNA, partially codon-optimized human cDNA, human MPZ polypeptide, human MPZ promoter, and human MPZ expression cassette of the disclosure.

TABLE 1
Human MPZ cDNA, 3′UTR DNA, partially codon-optimized human
cDNA, human MPZ polypeptide, human MPZ promoter, and human
MPZ expression cassette sequences.

| | | SEQ ID |
|---|---|---|
| Gene | Sequence | NO: |
| Human MPZ | ATGGCTCCTGGGGCTCCCTCATCCAGCCCCAGCCCTATCCTGGCTGTGCTGCTCTTCTCTTCTTT | 1 |
| cDNA | GGTGCTGTCCCCGGCCCAGGCCATCGTGGTTTACACCGACAGGGAGGTCCATGGTGCTGTGGGCT | |
| (747 bp, | CCCGGGTGACCCTGCACTGCTCCTTCTGGTCCAGTGAGTGGGTCTCAGATGACATCTCCTTCACC | |
| NM_000530.8) | TGGCGCTACCAGCCCGAAGGGGGCAGAGATGCCATTTCGATCTTCCACTATGCCAAGGGACAACC | |
| | CTACATTGACGAGGTGGGGACCTTCAAAGAGCGCATCCAGTGGGTAGGGGACCCTCGCTGGAAGG | |
| | ATGGCTCCATTGTCATACACAACCTAGACTACAGTGACAATGGCACGTTCACTTGTGACGTCAAA | |
| | AACCCTCCAGACATAGTGGGCAAGACCTCTCAGGTCACGCTGTATGTCTTTGAAAAAGTGCCAAC | |
| | TAGGTACGGGGTCGTTCTGGGAGCTGTGATCGGGGGTGTCCTCGGGGTGGTGCTGTTGCTGCTGC | |
| | TGCTTTTCTACGTGGTTCGGTACTGCTGGCTACGCAGGCAGGCGGCCCTGCAGAGGAGGCTCAGT | |
| | GCTATGGAGAAGGGGAAATTGCACAAGCCAGGAAAGGACGCGTCGAAGCGCGGGCGGCAGACGCC | |
| | AGTGCTGTATGCAATGCTGGACCACAGCAGAAGCACCAAAGCTGTCAGTGAGAAGAAGGCCAAGG | |
| | GGCTGGGGGAGTCTCGCAAGGATAAGAAATAG | |
| Human MPZ | CGGTTAGCGGGCCGGGGGGGGGATCGGGGGTTAGGGGGGAGTCCGCCAAAGGCCCAAAGGTGATG | 2 |
| 3′UTR | GTCATCGAGATGGAGCTACGAAAGGATGAGCAGAGCCCGGAGCTCCGGCCTGCTGTCAAGTCCCC | |
| (1141 bp, | CAGCAGAACCAGCCTCAAAAACGCCCTCAAGAACATGATGGGCCTGAACTCGGACAAGTGATCGC | |
| NM_000530.8): | CACCCCCCCACCCCAGGCCCTGCCAGAGCAGGGGGACCTAGGCTCCTCTTACCCCCGTCTAGGTG | |
| | CTTTCCCTCTTTGCTCCCCCGCCCTGCCCTGCCCTCACCTCCCTTTGAGATGTAAGTTTCATTCC | |
| | AGAATTCATTCCCCAGGCAATTGTATTCTCCCCCACCTTCACCCCTGGCTTTCTGGGAGCCCAGG | |
| | AGCTAATCCTACCCCTCACCTGCCCCGGGGGCTGTGTGTTTGGTGCCTGTCCACCTGAGCACTGA | |
| | GAAGAAAGGGACTTTGATACCCTCTGCCTCAAGTCCAGGCCACCTGGCATTCCCATCTCCTGCAT | |
| | CCCCCAGCCTGTCCCCCTGGCTGTTTCCTCTCCCGTCCCTCCCTCCCCTCTACCAGGTGGCCCAG | |
| | CTCCATACTCTGTCCCCCCAGCTAATACCCAGAGCACCCAGATCAGACTCTCCTTCAGGGTTTAT | |
| | TTAGGTTATTATTTTTTATTTTTTAATCCATTCTTTGTTTGTTTACCTGTGCTCATCCTCTGCCC | |
| | TTACACCCATGACTGAGGACCAATGACGTCATGTGGCTTTTGCAATTCACGCCCCCCTTAAGTCC | |
| | TTAATGAAGAGCCAGCCCAAGTAGAGGGGCCCCTGATCCTCACACTTCAGTATAGCATTGGTTCC | |
| | CCCTGACCACTTTGGAGCACTGTTCTGGGACTCCAGGTCTTGAGGAGAGAGACAGAGAGAGAGAA | |
| | TGGATCCTCATAGGTCAGGGAGTGGGGGAGGGGGCAAATGAGCCTTAAGAAATGGTTTTTAAACA | |
| | ACCAAACAAAAAGCAGGAAAAACAAATGGGAAATGGGGGGGGGGGGGGAGGAAGAGGCTGCACTG | |
| | CAGCCACAGGGGATTCTTAGGATTTTTCTACATTCTGTATATTTCTTCTCAAACCTCCAAATGTC | |
| | CTTAAATGTTTAATAAACACTGACATTTCCAGAA | |
| Partially codon | ATGGCTCCTGGGGCTCCCTCATCCAGCCCCAGCCCTATCCTGGCTGTGCTGCTCTTCTCTTCTTT | 3 |
| optimized MPZ | GGTGCTGTCCCCGGCCCAGGCCATCGTGGTTTACACCGACAGGGAGGTCCATGGTGCTGTGGGCT | |
| cDNA (coMPZ | CCCGGGTGACCCTGCACTGCTCCTTCTGGTCCAGTGAGTGGGTCTCAGATGACATCTCCTTCACC | |
| or resMPZ): | TGGCGCTACCAGCCCGAAGGGGGCAGAGACGCAATAAGCATATTTCATTATGCCAAGGGACAACC | |
| 225-247 (target | CTACATTGACGAGGTGGGGACCTTCAAAGAGCGCATCCAGTGGGTAGGGGACCCCAGGTGGAAAG | |
| site for miMPZ | ACGGTTCAATCGTCATACACAACCTAGACTACAGTGACAATGGCACGTTCACTTGTGACGTCAAA | |
| 225 and 226) | AACCCTCCAGACATAGTGGGCAAGACCTCTCAGGTCACGCTGTATGTCTTTGAAAAAGTGCCAAC | |
| 315-338 (target | TAGGTACGGGGTCGTTCTGGGAGCTGTGATCGGGGGTGTCCTCGGGGTGGTGCTGTTGCTGCTGC | |
| sites for miMPZ | TGCTTTTCTACGTGGTTCGGTACTGCTGGCTACGCAGGCAGGCGGCCCTGCAGAGGAGGCTCAGT | |
| 315, 316, 317) | GCTATGGAGAAGGGGAAATTGCACAAGCCAGGAAAGGACGCGTCGAAGCGCGGGCGGCAGACGCC | |
| 718-744 (target | AGTGCTGTATGCAATGCTGGACCACAGCAGAAGCACCAAAGCTGTCAGTGAGAAGAAGGCCAAGG | |
| site for miMPZ | GGCTTGGCGAAAGCAGGAAAGACAAAAAGTAG | |
| 718, 719, 720, | | |
| 721, 722, 723) | | |
| MPZ | MAPGAPSSSPSPILAVLLFSSLVLSPAQAIVVYTDREVHGAVGSRVTLHCSFWSSEWVSDDISFT | 4 |
| polypeptide | WRYQPEGGRDAISIFHYAKGQPYIDEVGTFKERIQWVGDPRWKDGSIVIHNLDYSDNGTFTCDVK | |
| sequence | NPPDIVGKTSQVTLYVFEKVPTRYGVVLGAVIGGVLGVVLLLLLLFYVVRYCWLRRQAALQRRLS | |
| (NM_000530.8) | AMEKGKLHKPGKDASKRGRQTPVLYAMLDHSRSTKAVSEKKAKGLGESRKDKK | |
| human MPZ | TGGCACAATCATAGCTCGCTGCAGCCTTGATTTCCCAGGTTCAAGCGATCTTCCTGCCTCAGCCT | 5 |
| promoter | CCCAAGTAGCTGGGACTACAAGCATGTGATAACATGCCCAGCTGATTTTTTAAAAAATTTTGGTA | |
| 1200 bp | GAGGCGAGGTCTTGCTATGTTGCCCAGACAGGTCTCAAACTCCTAAACTCAAGCAATCCTCCTGC | |
| (ENSG00000158887) | ATAAGCCTACCAAAGTGCTGAGATTACAGGTGTCAGCCACTATGCCTGGCATAAACTTCATTTAT | |
| | TAAAGTTTATTTTGTCTTTAATCTCTCATATAACTTAGTCTTCCTGATATTGCAGCTGTGTGTGC | |
| | CCCTCTTTTGTACTCCCAGCATTTTGTTCATTACTAAAGGAAGTGTCATGGCTTATTATACTTGA | |
| | TTGTTGATGGGTTTGTCCTCTGATCTTCCCATCTCCACCTCCCCAAACCAAATTTTCAACTCCTT | |
| | GCTGGAAGGACTTAATTTTTATTCCTCTCTCTATTACCTGCATTCTCATACTTTACATATTGCTG | |
| | GCACTTAATACAATTTTGTAGCCTTGAAATAAATTGAAATGGACTTAAACAGCAGCATGAAGCAC | |
| | TGAAGGACTTCTTGACAAACGGAAAGGTCAGGGGCTTCTTGCCTGGAAATAGTCCAGTGGAGAAA | |
| | AACTTCTGTCTGGGAAGAATCGCACAGGATGAAGGGAGGTGCGGGGAAAAAAACTCCCATAGGAC | |
| | TTGGTCATCTCAAGAAGTCTGTAATGCAGCCCACATTAGAGGAGATAACAGGGGATATCCTATTT | |
| | TCAGAGTTCTCTGGGGGAAACCTCCCTCTAGTTCCTAGGGCTGTGAGGCAGCCTCTCTCAGGCAA | |
| | GGAGGCTGAGGAGAAATCCCTTTTTATGGCCTTTAAATTGAGGTTCCATATCTATCCCTCAGAGA | |
| | AGTGTGTCTGTGTCCCTGTTTTTGTCCCTCTCCCTCACCACCCCCCACAACATTCCAGCCTGGGG | |
| | CAGGGGGAGGCCAGTGGACACAAAGCCCTCTGTGTATGGGGTGGTATGTGTCCCCCCACCCCTCC | |
| | ACCCAGACTATACAATGCCCCTTCTGCTCCCTGCACTCTGCCCCCCTCCCCACCACCTCTCAACT | |
| | GCACATGCCAGGCTGCAATTGGTTACTGGCTGAGGACAGCCCCCTCATGCTGGGGCCCTAGGGGA | |
| | TTTTAAGCAGGTTCCAGGAACCCCCCGTTC | |
| MPZ | TCTAGAaggcctGTCGACggcgccTGGCACAATCATAGCTCGCTGCAGCCTTGATTTCCCAGGTT | 6 |
| expression | CAAGCGATCTTCCTGCCTCAGCCTCCCAAGTAGCTGGGACTACAAGCATGTGATAACATGCCCAG | |
| cassette | CTGATTTTTTAAAAAATTTTGGTAGAGGCGAGGTCTTGCTATGTTGCCCAGACAGGTCTCAAACT | |
| Comprises | CCTAAACTCAAGCAATCCTCCTGCATAAGCCTACCAAAGTGCTGAGATTACAGGTGTCAGCCACT | |
| 1200 bp of | ATGCCTGGCATAAACTTCATTTATTAAAGTTTATTTTGTCTTTAATCTCTCATATAACTTAGTCT | |
| human MPZ | TCCTGATATTGCAGCTGTGTGTGCCCCTCTTTTGTACTCCCAGCATTTTGTTCATTACTAAAGGA | |
| promoter | AGTGTCATGGCTTATTATACTTGATTGTTGATGGGTTTGTCCTCTGATCTTCCCATCTCCACCTC | |
| (ENSG0000015 | CCCAAACCAAATTTTCAACTCCTTGCTGGAAGGACTTAATTTTTATTCCTCTCTCTATTACCTGC | |
| 8887), | ATTCTCATACTTTACATATTGCTGGCACTTAATACAATTTTGTAGCCTTGAAATAAATTGAAATG | |
| 63bp human | GACTTAAACAGCAGCATGAAGCACTGAAGGACTTCTTGACAAACGGAAAGGTCAGGGGCTTCTTG | |
| MPZ 5′UTR, | CCTGGAAATAGTCCAGTGGAGAAAAACTTCTGTCTGGGAAGAATCGCACAGGATGAAGGGAGGTG | |
| and | CGGGGAAAAAAACTCCCATAGGACTTGGTCATCTCAAGAAGTCTGTAATGCAGCCCACATTAGAG | |
| 747 bp partially | GAGATAACAGGGGATATCCTATTTTCAGAGTTCTCTGGGGGAAACCTCCCTCTAGTTCCTAGGGC | |
| codon | TGTGAGGCAGCCTCTCTCAGGCAAGGAGGCTGAGGAGAAATCCCTTTTTATGGCCTTTAAATTGA | |
| optimized MPZ | GGTTCCATATCTATCCCTCAGAGAAGTGTGTCTGTGTCCCTGTTTTTGTCCCTCTCCCTCACCAC | |
| cDNA | CCCCCACAACATTCCAGCCTGGGGCAGGGGGAGGCCAGTGGACACAAAGCCCTCTGTGTATGGGG | |
| | TGGTATGTGTCCCCCCACCCCTCCACCCAGACTATACAATGCCCCTTCTGCTCCCTGCACTCTGC | |
| | CCCCCTCCCCACCACCTCTCAACTGCACATGCCAGGCTGCAATTGGTTACTGGCTGAGGACAGCC | |
| | CCCTCATGCTGGGGCCCTAGGGGATTTTAAGCAGGTTCCAGGAACCCCCCGTTCGCTAGCAGTTC | |
| | CTGGTCCCCCACTTTCTCAACCCCACAGATGCTCCGGGCCCCTGCCCCTGCCCCAGCTatggctc | |
| | ctggggctccctcatccagccccagccctatcctggctgtgctgctcttctcttctttggtgctg | |
| | tccccggcccaggccatcgtggtttacaccgacagggaggtccatggtgctgtgggctcccgggt | |
| | gaccctgcactgctccttctggtccagtgagtgggtctcagatgacatctccttcacctggcgct | |
| | accagcccgaagggggcagagacgcaataagcatatttcattatgccaagggacaaccctacatt | |
| | gacgaggggggaccttcaaagagcgcatccagtgggtaggggaccccaggtggaaagacggttca | |
| | atcgtcatacacaacctagactacagtgacaatggcacgttcacttgtgacgtcaaaaaccctcc | |
| | agacatagtgggcaagacctctcaggtcacgctgtatgtctttgaaaaagtgccaactaggtacg | |
| | gggtcgttctgggagctgtgatcgggggtgtcctcggggtggtgctgttgctgctgctgcttttc | |
| | tacgtggttcggtactgctggctacgcaggcaggcggccctgcagaggaggctcagtgctatgga | |
| | gaaggggaaattgcacaagccaggaaaggacgcgtcgaagcgcgggcggcagacgccagtgctgt | |
| | atgcaatgctggaccacagcagaagcaccaaagctgtcagtgagaagaaggccaaggggcttggc | |
| | gaaagcaggaaagacaaaaagtagACTAGTcatatgAACTTGTTTATTGCAGCTTATAATGGTTA | |
| | CAAATAAAGCAATAGCATCACAAATTTCACAAATAAAGCATTTTTTTCACTGCATTCTAGTTGTG | |
| | GTTTGTCCAAACTCATCAATGTATCTTATCATGTCTGttaattaaCATGGTGATGCGGTTTTGGC | |
| | AGTACATCAATGGGCGTGGATAGCGGTTTGACTCACGGGGATTTCCAATGCATgcggccgcTCTA | |
| | GA | |
| Mouse Mpz | ATGGCTCCCGGGGCTCCCTCCTCCAGCCCCAGCCCTATCCTGGCTGCCCTGCTCTTCTCTTCTTT | 83 |
| CDNA (747 bp, | GGTGCTCTCTCCAGCCCTGGCCATTGTGGTTTACACGGACAGGGAAATCTATGGTGCCGTGGGCT | |
| NM_001315500.1) | CCCAGGTGACCCTGCACTGCTCCTTCTGGTCCAGTGAATGGGTCTCAGATGACATCTCTTTTACC | |
| | TGGCGCTACCAGCCTGAAGGGGGCCGAGATGCCATTTCGATCTTCCACTATGCCAAGGGACAACC | |
| | TTACATCGATGAGGTGGGGACCTTCAAAGAGCGCATCCAGTGGGTAGGGGACCCTCGCTGGAAGG | |
| | ATGGCTCCATTGTCATACACAACCTAGACTACAGTGACAACGGCACTTTCACATGTGATGTCAAA | |
| | AACCCACCGGACATAGTGGGCAAGACCTCTCAGGTCACGCTCTATGTCTTTGAAAAAGTGCCCAC | |
| | TAGGTATGGGGTGGTGTTGGGAGCAGTGATCGGGGGCATCCTCGGGGTGGTGCTGTTGCTGCTGT | |
| | TGCTCTTCTACCTGATTCGGTACTGCTGGCTGCGCAGGCAGGCTGCCCTGCAGAGAAGGCTCAGT | |
| | GCCATGGAGAAGGGGAGATTTCACAAATCTTCGAAGGACTCCTCGAAGCGAGGGCGGCAGACGCC | |
| | AGTGCTGTATGCCATGCTGGACCACAGCCGAAGCACCAAAGCTGCCAGTGAGAAGAAATCAAAAG | |
| | GGCTGGGGGAGTCTCGCAAGGATAAGAAATAG | |
| 3′ UTR of | CGGTTAGCGGGCCGGGCGGGGGGTCGGGGGTCTGCGACGGAGTCCTCCAAAGGCTCTCAGGTGGT | 84 |
| mouse Mpz | GGTCATCGAGATGGAGCTTCGCAAAGATGAGCAGAGCTCGGAGCTCCGGCCTGCAGTCAAGTCCC | |
| gene (1087 bp, | CCAGTAGAACCAGCCTCAAGAACGCCCTCAAGAACATGATGGGCCTGGACTCGGACAAGTGACCG | |
| NM_001315500.1) | TCACATCCAGGCCCTGTCAGAGCAGGGGACCTAGGCTCCTCTTTTTCCCGGGTCTAGGTGCTTTC | |
| | CTCTTGCTCCTCAGCCCTGCCCTGCCCTTACCTCCCAATGAGATGTAAAGTTTCATTCCAACCTT | |
| | CCTTTCCAAGTCTTGTACTCCTCCTTCGCCTTTACCCCTCGGCTTTCCAGGAGCCTAGGGACTAA | |
| | CCCTACTCCTCGCCTGCCCCAAGGGCTGTGTGTTTGGTGCCTGTCTCTCAGACACCGAGAAGAAA | |
| | GGGACCTTGATAGCCTCTGCCCAACTCCAGGCCACTTGGCCTTCCCATCTCTCCCCCCCCCGCCC | |
| | CCCCCCCCAGCTGATCCCCTGGCTCCTTCCTGCTCTCCCTTTCCTACCTTCTGGTGGCCCAGCTC | |
| | CACACTCTGTCCTCGGGCTCAAATCCACATCGTGCAGAGCAGACTCTCCATCAGGGTTTATTTAG | |
| | GTTGCTGAGTATTTTTTATTTGCCCATCCATTCTTTCGTTTGTTTACCTGAGCCCACGGCCCCAT | |
| | GACCGAGGACCAATGACGTCATGTGGCTTTTGCAGTTCCCCACTAAGTCTTTACTGGAGAGCCAG | |
| | GCAGGCAAGCAGCGCATAATCGATGCCCTCTGACCACTACGGAGCACAGTTCTGGGACTCCGAGA | |
| | TTTGGGGAAGGAAAGAAAAAAAAATGGATCCTCACAGGTCAGGGGTGCAGTAGGGGGCTTTAAGA | |
| | AATGTCTTCAAAATAAATAATCAGAACAGGAAACGCCAGCGAGAATGGGAAGGAAAGGAAGTGGC | |
| | TACACCCCCGGGCACAGTGGACTGTTAGGACTTTTCTACATTCTGTATATTTCTCCTACCATCTC | |
| | CAAATGTCCTTAAATGTTTAATAAACACTGACATTTCCAGAAGCTGC | |

The disclosure includes the use of RNA interference to inhibit expression mutant MPZ to ameliorate and/or treat subjects with diseases or disorders resulting from the mutated MPZ gene and the resultant altered version of mRNA. RNA interference (RNAi) is a mechanism of gene regulation in eukaryotic cells that has been considered for the treatment of various diseases. RNAi refers to post-transcriptional control of gene expression mediated by inhibitory RNAs. The miRNAs are small (21-25 nucleotides), noncoding RNAs that share sequence homology and base-pair with 3′ untranslated regions of cognate messenger RNAs (mRNAs). The interaction between the miRNAs and mRNAs directs cellular gene silencing machinery to prevent the translation of the mRNAs.

As an understanding of natural RNAi pathways has developed, researchers have designed artificial shRNAs and snRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. shRNA and miRNA are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005). Importantly, this vector-expressed approach leverages the decades-long advancements already made in the muscle gene therapy field, but instead of expressing protein coding genes, the vector cargo in RNAi therapy strategies are artificial shRNA or miRNA cassettes targeting disease genes-of-interest. This strategy is used to express a natural miRNA. Each shRNA/miRNA is based on hsa-miR-30a sequences and structure. The natural mir-30a mature sequences are replaced by unique sense and antisense sequences derived from the target gene.

The disclosure provides specifically designed artificial miRNAs to silence endogenous mutant and wild-type MPZ expression. The disclosure also provides specifically designed artificial miRNAs to silence endogenous mutant and wild-type MPZ expression, while simultaneously replacing wild-type MPZ expression with a miRNA-resistant (rMPZ) gene. This “knockdown and replace” strategy reduces defective MPZ protein and replaces the defective protein with healthy MPZ protein. In some aspects, the artificial MPZ-reducing miRNA is delivered alone. In some aspects, the artificial MPZ-reducing miRNA is delivered with the rMPZ gene to neuronal cells including, but not limited to, Schwann cells. The artificial miRNAs (called miMPZ herein) were designed to specifically and equally target both human MPZ and mouse Mpz genes, with no predicted non-specific binding to other transcripts. This strategy allows the translation of results from murine models to human clinical trials. Thus, the miMPZ described and disclosed as therapeutics for treating MPZ mutations and diseases associated with those MPZ mutations including, but not limited to, CMT disease.

The disclosure provides nucleic acids comprising polynucleotides encoding inhibitory RNAs (microRNA (miRNA)) targeting MPZ to knock down, inhibit, or prevent the expression of the MPZ gene and protein, including mutant MPZ gene and protein. The inhibitory RNAs comprise antisense sequences, which inhibit the expression of the MPZ gene. In some aspects, the disclosure provides nucleic acids comprising polynucleotides encoding MPZ miRNAs, and RNAi-resistant MPZ genes. The disclosure provides full-length un-processed MPZ miRNAs and mature or process miRNAs. The disclosure also provides MPZ sequences that the miRNA sequences are designed to target.

In some aspects, the disclosure provides a nucleic acid comprising a polynucleotide encoding a MPZ miRNA comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18.

In some aspects, the disclosure provides a nucleic acid comprising a polynucleotide encoding a MPZ miRNA comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18.

In some aspects, the disclosure provides a nucleic acid comprising a polynucleotide comprising an MPZ miRNA comprising a full length miRNA antisense guide strand comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 19-30. Thus, in some aspects, the disclosure provides a nucleic acid comprising a polynucleotide comprising or encoding an MPZ final processed guide strand miRNA comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 31-42. In some more particular aspects, therefore, the disclosure provides a nucleic acid comprising a polynucleotide sequence encoding an MPZ final processed guide strand miRNA comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide in any one of SEQ ID NOs: 31-42.

In some aspects, the disclosure provides a nucleic acid comprising a polynucleotide targeting a DNA sequence of the MPZ gene comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 43-54.

Table 2 set out below provides exemplary miRNA encoding sequences, RNA sequences, and MPZ target sequences. Table 3 set out below provides PCR primers (SEQ ID NOs: 55-78) used in synthesizing DNA encoding the microRNAs (miRNAs).

TABLE 2
miRNA encoding sequences, full-length RNA sequences,
mature RNA sequences, and MPZ target sequences.

| | | DNA Sequence | | | | Mature RNA | | Target DNA | |
|---|---|---|---|---|---|---|---|---|---|
| | | encoding | SEQ | RNA sequence | SEQ | sequence | SEQ | sequence on | SEQ |
| | | the miRNA | ID | full length | ID | (22bp), | ID | the MPZ gene | ID |
| No. | miMPZ | 5′ to 3′ | NO: | 5′ to 3′ | NO: | 5′ to 3′ | NO: | 5′ to 3′ | NO: |
| 1 | 225 | CTCGAGTGAGCGCGC | 7 | CUCGAGUGAGCGCGC | 19 | AUAGUGGAAGA | 31 | TGCCATTTCGA | 43 |
| | | CATTTOGATCTTCCA | | CAUUUCGAUCUUCCA | | UCGAAAUGGCA | | TCTTCCACTAT | |
| | | CTATCTGTAAAGCCA | | CUAUCUGUAAAGCCA | | | | | |
| | | CAGATGGGATAGTGG | | CAGAUGGGAUAGUGG | | | | | |
| | | AAGATCGAAATGGCA | | AAGAUCGAAAUGGCA | | | | | |
| | | TGCCTACTAGT | | UGCCUACUAGU | | | | | |
| 2 | 226 | CTCGAGTGAGCGACC | 8 | CUCGAGUGAGCGACC | 20 | CAUAGUGGAAG | 32 | GCCATTTCGAT | 44 |
| | | ATTTCGATCTTCCAC | | AUUUCGAUCUUCCAC | | AUCGAAAUGGC | | CTTCCACTATG | |
| | | TATGCTGTAAAGCCA | | UAUGCUGUAAAGCCA | | | | | |
| | | CAGATGGGCATAGTG | | CAGAUGGGCAUAGUG | | | | | |
| | | GAAGATCGAAATGGC | | GAAGAUCGAAAUGGC | | | | | |
| | | TGCCTACTAGT | | UGCCUACUAGU | | | | | |
| 3 | 315 | CTCGAGTGAGCGCCG | 9 | CUCGAGUGAGCGCCG | 21 | AAUGGAGCCAU | 33 | TCGCTGGAAGG | 45 |
| | | CTGGAAGGATGGCTC | | CUGGAAGGAUGGCUC | | CCUUCCAGCGA | | ATGGCTCCATT | |
| | | CATTCTGTAAAGCCA | | CAUUCUGUAAAGCCA | | | | | |
| | | CAGATGGGAATGGAG | | CAGAUGGGAAUGGAG | | | | | |
| | | CCATCCTTCCAGCGA | | CCAUCCUUCCAGCGA | | | | | |
| | | TGCCTACTAGT | | UGCCUACUAGU | | | | | |
| 4 | 316 | CTCGAGTGAGCGAGC | 10 | CUCGAGUGAGCGAGC | 22 | CAAUGGAGCCA | 34 | CGCTGGAAGGA | 46 |
| | | TGGAAGGATGGCTCC | | UGGAAGGAUGGCUCC | | UCCUUCCAGCG | | TGGCTCCATTG | |
| | | TTGCTGTAAAGCCAC | | UUGCUGUAAAGCCAC | | | | | |
| | | AGATGGGCAATGGAG | | AGAUGGGCAAUGGAG | | | | | |
| | | CCATCCTTCCAGCGT | | CCAUCCUUCCAGCGU | | | | | |
| | | GCCTACTAGT | | GCCUACUAGU | | | | | |
| 5 | 317 | CTCGAGTGAGCGACT | 11 | CUCGAGUGAGCGACU | 23 | ACAAUGGAGCC | 35 | GCTGGAAGGAT | 47 |
| | | GGAAGGATGGCTCCA | | GGAAGGAUGGCUCCA | | AUCCUUCCAGC | | GGCTCCATTGT | |
| | | TTGTCTGTAAAGCCA | | UUGUCUGUAAAGCCA | | | | | |
| | | CAGATGGGACAATGG | | CAGAUGGGACAAUGG | | | | | |
| | | AGCCATCCTTCCAGC | | AGCCAUCCUUCCAGC | | | | | |
| | | TGCCTACTAGT | | UGCCUACUAGU | | | | | |
| 6 | 718 | CTCGAGTGAGCGATG | 12 | CUCGAGUGAGCGAUG | 24 | UAUCCUUGCGA | 36 | CTGGGGGAGTC | 48 |
| | | GGGGAGTCTCGCAAG | | GGGGAGUCUCGCAAG | | GACUCCCCCAG | | TCGCAAGGATA | |
| | | GATACTGTAAAGCCA | | GAUACUGUAAAGCCA | | | | | |
| | | CAGATGGGTATCCTT | | CAGAUGGGUAUCCUU | | | | | |
| | | GCGAGACTCCCCCAG | | GCGAGACUCCCCCAG | | | | | |
| | | TGCCTACTAGT | | UGCCUACUAGU | | | | | |
| 7 | 719 | CTCGAGTGAGCGCGG | 13 | CUCGAGUGAGCGCGG | 25 | UUAUCCUUGCG | 37 | TGGGGGAGTCT | 49 |
| | | GGGAGTCTCGCAAGG | | GGGAGUCUCGCAAGG | | AGACUCCCCCA | | CGCAAGGATAA | |
| | | ATAACTGTAAAGCCA | | AUAACUGUAAAGCCA | | | | | |
| | | CAGATGGGTTATCCT | | CAGAUGGGUUAUCCU | | | | | |
| | | TGCGAGACTCCCCCA | | UGCGAGACUCCCCCA | | | | | |
| | | TGCCTACTAGT | | UGCCUACUAGU | | | | | |
| 8 | 720 | CTCGAGTGAGCGAGG | 14 | CUCGAGUGAGCGAGG | 26 | CUUAUCCUUGC | 38 | GGGGGAGTCTC | 50 |
| | | GGAGTCTCGCAAGGA | | GGAGUCUCGCAAGGA | | GAGACUCCCCC | | GCAAGGATAAG | |
| | | TAAGCTGTAAAGCCA | | UAAGCUGUAAAGCCA | | | | | |
| | | CAGATGGGCTTATCC | | CAGAUGGGCUUAUCC | | | | | |
| | | TTGCGAGACTCCCCC | | UUGCGAGACUCCCCC | | | | | |
| | | TGCCTACTAGT | | UGCCUACUAGU | | | | | |
| 9 | 721 | CTCGAGTGAGCGAGG | 15 | CUCGAGUGAGCGAGG | 27 | UCUUAUCCUUG | 39 | GGGGAGTCTCG | 51 |
| | | GAGTCTCGCAAGGAT | | GAGUCUCGCAAGGAU | | CGAGACUCCCC | | CAAGGATAAGA | |
| | | AAGACTGTAAAGCCA | | AAGACUGUAAAGCCA | | | | | |
| | | CAGATGGGTCTTATC | | CAGAUGGGUCUUAUC | | | | | |
| | | CTTGCGAGACTCCCC | | CUUGCGAGACUCCCC | | | | | |
| | | TGCCTACTAGT | | UGCCUACUAGU | | | | | |
| 10 | 722 | CTCGAGTGAGCGAGG | 16 | CUCGAGUGAGCGAGG | 28 | UUCUUAUCCUU | 40 | GGGAGTCTCGC | 52 |
| | | AGTCTCGCAAGGATA | | GAGUCUCGCAAGGAU | | GCGAGACUCCC | | AAGGATAAGAA | |
| | | AGAACTGTAAAGCCA | | AAGACUGUAAAGCCA | | | | | |
| | | CAGATGGGTTCTTAT | | CAGAUGGGUCUUAUC | | | | | |
| | | CCTTGCGAGACTCCC | | CUUGCGAGACUCCCC | | | | | |
| | | TGCCTACTAGT | | UGCCUACUAGU | | | | | |
| 11 | 723 | CTCGAGTGAGCGAGA | 17 | CUCGAGUGAGCGAGA | 29 | UUUCUUAUCCU | 41 | GGAGTCTCGCA | 53 |
| | | GTCTCGCAAGGATAA | | GUCUCGCAAGGAUAA | | UGCGAGACUCC | | AGGATAAGAAA | |
| | | GAAACTGTAAAGCCA | | GAAACUGUAAAGCCA | | | | | |
| | | CAGATGGGTTTCTTA | | CAGAUGGGUUUCUUA | | | | | |
| | | TCCTTGCGAGACTCC | | UCCUUGCGAGACUCC | | | | | |
| | | TGCCTACTAGT | | UGCCUACUAGU | | | | | |
| 12 | 1852 | CTCGAGTGAGCGATC | 18 | CUCGAGUGAGCGAUC | 30 | UGUUUAUUAAA | 42 | GTCCTTAAATG | 54 |
| | | CTTAAATGTTTAATA | | CUUAAAUGUUUAAUA | | CAUUUAAGGAC | | TTTAATAAACA | |
| | | AACACTGTAAAGCCA | | AACACUGUAAAGCCA | | | | | |
| | | CAGATGGGTGTTTAT | | CAGAUGGGUGUUUAU | | | | | |
| | | TAAACATTTAAGGAC | | UAAACAUUUAAGGAC | | | | | |
| | | TGCCTACTAGT | | UGCCUACUAGU | | | | | |

TABLE 3
PCR primers for DNA encoding the microRNAs (miRNAs).

| | | SEQ ID |
|---|---|---|
| miMPZ No. | Sequence (5′ to 3′) | NO: |
| 1-miMPZ_225 | | |
| F primer | AAAACTCGAGTGAGCGCGCCATTTCGATCTTCCACTATCTGTAAAGCCACAGATGGG | 55 |
| R primer | TTTTACTAGTAGGCATGCCATTTCGATCTTCCACTATCCCATCTGTGGCTTTACAG | 56 |
| 2-miMPZ_226 | | |
| F primer | AAAACTCGAGTGAGCGACCATTTCGATCTTCCACTATGCTGTAAAGCCACAGATGGG | 57 |
| R primer | TTTTACTAGTAGGCAGCCATTTCGATCTTCCACTATGCCCATCTGTGGCTTTACAG | 58 |
| 3-miMPZ_315 | | |
| F primer | AAAACTCGAGTGAGCGCCGCTGGAAGGATGGCTCCATTCTGTAAAGCCACAGATGGG | 59 |
| R primer | TTTTACTAGTAGGCATCGCTGGAAGGATGGCTCCATTCCCATCTGTGGCTTTACAG | 60 |
| 4-miMPZ_316 | | |
| F primer | AAAACTCGAGTGAGCGAGCTGGAAGGATGGCTCCATTGCTGTAAAGCCACAGATGGG | 61 |
| R primer | TTTTACTAGTAGGCACGCTGGAAGGATGGCTCCATTGCCCATCTGTGGCTTTACAG | 62 |
| 5-miMPZ_317 | | |
| F primer | AAAACTCGAGTGAGCGACTGGAAGGATGGCTCCATTGTCTGTAAAGCCACAGATGGG | 63 |
| R primer | TTTTACTAGTAGGCAGCTGGAAGGATGGCTCCATTGTCCCATCTGTGGCTTTACAG | 64 |
| 6-miMPZ_718 | | |
| F primer | AAAACTCGAGTGAGCGATGGGGGAGTCTCGCAAGGATACTGTAAAGCCACAGATGGG | 65 |
| R primer | TTTTACTAGTAGGCACTGGGGGAGTCTCGCAAGGATACCCATCTGTGGCTTTACAG | 66 |
| 7-miMPZ_719 | | |
| F primer | AAAACTCGAGTGAGCGCGGGGGAGTCTCGCAAGGATAACTGTAAAGCCACAGATGGG | 67 |
| R primer | TTTTACTAGTAGGCATGGGGGAGTCTCGCAAGGATAACCCATCTGTGGCTTTACAG | 68 |
| 8-miMPZ_720 | | |
| F primer | AAAACTCGAGTGAGCGAGGGGAGTCTCGCAAGGATAAGCTGTAAAGCCACAGATGGG | 69 |
| R primer | TTTTACTAGTAGGCAGGGGGAGTCTCGCAAGGATAAGCCCATCTGTGGCTTTACAG | 70 |
| 9-miMPZ_721 | | |
| F primer | AAAACTCGAGTGAGCGAGGGAGTCTCGCAAGGATAAGACTGTAAAGCCACAGATGGG | 71 |
| R primer | TTTTACTAGTAGGCAGGGGAGTCTCGCAAGGATAAGACCCATCTGTGGCTTTACAG | 72 |
| 10-miMPZ_722 | | |
| F primer | AAAACTCGAGTGAGCGAGGAGTCTCGCAAGGATAAGAACTGTAAAGCCACAGATGGG | 73 |
| R primer | TTTTACTAGTAGGCAGGGAGTCTCGCAAGGATAAGAACCCATCTGTGGCTTTACAG | 74 |
| 11-miMPZ_723 | | |
| F primer | AAAACTCGAGTGAGCGAGAGTCTCGCAAGGATAAGAAACTGTAAAGCCACAGATGGG | 75 |
| R primer | TTTTACTAGTAGGCAGGAGTCTCGCAAGGATAAGAAACCCATCTGTGGCTTTACAG | 76 |
| 12-miMPZ_1852 | | |
| F primer | AAAACTCGAGTGAGCGATCCTTAAATGTTTAATAAACACTGTAAAGCCACAGATGGG | 77 |
| R primer | TTTTACTAGTAGGCAGTCCTTAAATGTTTAATAAACACCCATCTGTGGCTTTACAG | 78 |

In some aspects, a nucleic acid of the disclosure comprises a sequence which is operatively linked to a transcriptional control element (including, but not limited to, a promoter, enhancer and/or a polyadenylation signal) that that is functional in the target cell. In some aspects, the polynucleotide (or nucleotide) sequence is linked to a promoter.

Inducible promoters are also contemplated. Non-limiting examples of inducible promoters include, but are not limited to a metallothionine promoter, a glucocorticoid promoter, a progesterone promoter, and a tetracycline-regulated promoter. The gene cassette comprising the MPZ replacement gene may also include intron sequences to facilitate processing of the MPZ RNA transcript when expressed in mammalian cells.

In some aspects, the polynucleotide sequence encoding the MPZ miRNA and/or the polynucleotide sequence encoding the replacement MPZ gene is expressed under the same promoter or each polynucleotide sequence is expressed under their own promoter. Such promoter(s) includes, but is not limited to, a U6 promoter, a U7 promoter, an H19 promoter, a neuron-specific promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, the creatine kinase promoter, a neuron-specific promoter, a myelin-specific promoter, or a native promoter. In some aspects, the promoter may be a Schwann cell-specific promoter or a myelin-specific promoter, including but limited to, those promoters described, for example, in WO 2020/245169. In some aspects, the promoter may be a mouse, rat, or human promoter. In some aspects, the nucleotide sequence encoding the MPZ miRNA and/or the replacement MPZ gene is expressed under a U6 promoter, a U7 promoter, an H19 promoter, or a neuron-specific promoter. In some aspects, such neuron-specific promoter is an MPZ promoter, a non-compact myelin associated protein (NCMPA or MP11) promoter, a PMP22 promoter, an MBP promoter, a SOX10 promoter, or a GAP43 promoter. In some aspects, the promoter used with the miMPZ is a U6 promoter. In some aspects, the promoter used with the MPZ replacement gene is an MPZ promoter or a mini-MPZ promoter. In some aspects, the MPZ promoter or mini-MPZ promoter is a mouse, rat, or human promoter. In MPZ promoter or mini-MPZ promoter is a human MPZ promoter or a human mini-MPZ promoter. In some aspects, the human MPZ promoter comprises the nucleotide sequence set out in SEQ ID NO: 5, or a variant thereof comprising at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5.

In some aspects, the products, methods, and uses of the disclosure also comprise short hairpin RNA or small hairpin RNA (shRNA) to affect MPZ expression (e.g., knockdown or inhibit expression). A short hairpin RNA (shRNA/Hairpin Vector) is an artificial RNA molecule with a tight hairpin turn that can be used to silence target gene expression via RNA interference (RNAi). shRNA is an advantageous mediator of RNAi in that it has a relatively low rate of degradation and turnover, but it requires use of an expression vector. Once the vector has transduced the host genome, the shRNA is then transcribed in the nucleus by polymerase II or polymerase III, depending on the promoter choice. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing. In some aspects, the disclosure includes the production and administration of an AAV vector expressing MPZ antisense sequences via shRNA. The expression of shRNAs is regulated by the use of various promoters. The promoter choice is essential to achieve robust shRNA expression. In various aspects, polymerase II promoters, such as U6 and H1, and polymerase III promoters are used. In some aspects, U6 shRNAs are used.

Thus, in some aspects, the disclosure uses U6 shRNA molecules to further inhibit, knockdown, or interfere with MPZ gene expression. Traditional small/short hairpin RNA (shRNA) sequences are usually transcribed inside the cell nucleus from a vector containing a Pol III promoter such as U6. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002)] because (1) the promoter is recognized by RNA polymerase III (poly III) and controls high-level, constitutive expression the promoter is active in most mammalian cell types. In some aspects, the promoter is a type III Pol III promoter in that all elements required to control expression of the shRNA are located upstream of the transcription start site (Paule et al., Nucleic Acids Res. 28(6):1283-98 (2000)). The disclosure includes both murine and human U6 promoters. The shRNA containing the sense and antisense sequences from a target gene connected by a loop is transported from the nucleus into the cytoplasm where Dicer processes it into small/short interfering RNAs (siRNAs).

In some embodiments, the products, methods, and uses of the disclosure comprise small nuclear ribonucleic acids (snRNAs), also commonly referred to as U-RNAs, to knockdown or further inhibit MPZ gene expression. snRNAs are a class of small RNA molecules that are found within the splicing speckles and Cajal bodies of the cell nucleus in eukaryotic cells. Small nuclear RNAs are associated with a set of specific proteins, and the complexes are referred to as small nuclear ribonucleoproteins (snRNP, often pronounced “snurps”). Each snRNP particle is composed of a snRNA component and several snRNP-specific proteins (including Sm proteins, a family of nuclear proteins). The snRNAs, along with their associated proteins, form ribonucleoprotein complexes (snRNPs), which bind to specific sequences on the pre-mRNA substrate. They are transcribed by either RNA polymerase II or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11, and U12. Sm-class snRNA are transcribed by RNA polymerase II. The second class, known as Lsm-class snRNA, consists of U6 and U6atac. Lsm-class snRNAs are transcribed by RNA polymerase III and never leave the nucleus, in contrast to Sm-class snRNA. In some aspects, the disclosure includes the production and administration of an AAV vector comprising U7 snRNA for the delivery of MPZ antisense sequences.

In some aspects, the disclosure uses U7 snRNA molecules to further inhibit, knockdown, or interfere with MPZ gene expression. U7 snRNA is normally involved in histone pre-mRNA 3′ end processing but, in some aspects, is converted into a versatile tool for splicing modulation or as antisense RNA that is continuously expressed in cells [Goyenvalle et al., Science 306(5702): 1796-9 (2004)]. By replacing the wild-type U7 Sm binding site with a consensus sequence derived from spliceosomal snRNAs, the resulting RNA assembles with the seven Sm proteins found in spliceosomal snRNAs. As a result, this U7 Sm OPT RNA accumulates more efficiently in the nucleoplasm and will no longer mediate histone pre-mRNA cleavage, although it can still bind to histone pre-mRNA and act as a competitive inhibitor for wild-type U7 snRNPs. By further replacing binding to the histone downstream element with one complementary to a particular target in a splicing substrate, it is possible to create U7 snRNAs capable of modulating specific splicing events. The advantage of using U7 derivatives is that the antisense sequence is embedded into a small nuclear ribonucleoprotein (snRNP) complex. Moreover, when embedded into a gene therapy vector, these small RNAs can be permanently expressed inside the target cell after a single injection [Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)].

U7 snRNA is normally involved in histone pre-mRNA 3′ end processing, but also is used as a versatile tool for splicing modulation or as antisense RNA that is continuously expressed in cells. One advantage of using U7 derivatives is that the antisense sequence is embedded into a small nuclear ribonucleoprotein (snRNP) complex. Moreover, when embedded into a gene therapy vector, these small RNAs can be permanently expressed inside the target cell after a single injection.

In some aspects, the disclosure includes a nanoparticle, extracellular vesicle, exosome, or vector comprising any of the nucleic acids of the disclosure or a combination of any one or more thereof. In some aspects, one or more copies of these sequences are combined into a single nanoparticle, extracellular vesicle, exosome, or vector.

The disclosure therefore includes vectors comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

In some embodiments, the disclosure utilizes AAV to deliver inhibitory RNAs, such as DNA encoding MPZ miRNA, which target the MPZ mRNA to inhibit mutant MPZ expression. AAV is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the AAV-4 is provided in GenBank Accession No. NC_001829; the AAV-5 genome is pro-vided in GenBank Accession No. AF085716; the complete genome of AAV-6 is provided in GenBank Accession No. NC_00 1862; at least portions of AAV-7 and AAV-8 genomes are pro-vided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromo-some integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Curr Topics in Microbiol and Immunol, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus, making cold preservation of AAV less critical. AAV may be lyophilized and AAV-infected cells are n superinfection. In some aspects, AAV is used to deliver the nucleic acid encoding the inhibitory RNA under the control of a U6 promoter, a U7 promoter, or a neuron-specific promoter. In some aspects, AAV is used to deliver the nucleic acid encoding the inhibitory RNA under the control of an MPZ promoter or a mini-MPZ promoter. In some aspects, the MPZ promoter or mini-MPZ promoter is mouse, rat, or human. In some aspects, AAV is used to deliver the nucleic acid encoding an RNAi-resistant replacement MPZ gene under the control of a U6 promoter, a U7 promoter, or a neuron-specific promoter. In some aspects, AAV is used to deliver the nucleic acid encoding an RNAi-resistant replacement MPZ gene under the control of an MPZ promoter or a mini-MPZ promoter. In some aspects, the MPZ promoter or mini-MPZ promoter is a mouse, rat, or human promoter. In some aspects, the MPZ promoter or mini-MPZ promoter is a human promoter.

In some embodiments, the AAV lacks rep and cap genes. In some embodiments, the AAV is a recombinant linear AAV (rAAV), a single-stranded AAV, or a recombinant self-complementary AAV (scAAV).

Advances in AAV vectors have led to safer and more efficient viral vehicles to deliver therapeutic transgenes in a single injection, and gene therapy is now a favorable therapeutic intervention for monogenic diseases. AAV vectors can provide long-term expression of gene products in post-mitotic target tissues. Thus, current AAV-based strategies may only require one-time vector administration.

Recombinant AAV genomes of the disclosure comprise one or more AAV ITRs flanking a polynucleotide encoding, for example, one or more MPZ inhibitory RNAs or MPZ miRNAs. The genomes of the rAAV provided herein either further comprise an RNAi-resistant replacement MPZ gene, or the RNAi-resistant replacement MPZ gene is present in a separate rAAV. The miRNA- and replacement MPZ-encoding polynucleotides are operatively linked to transcriptional control DNAs, for example promoter DNAs, which are functional in a target cell. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

Provided herein are rAAV, each encoding one or more MPZ miRNA and/or one or more RNAi-resistant replacement MPZ gene. The rAAV may encode one or more MPZ miRNAs and RNAi-resistant replacement MPZ genes. An rAAV encoding one or more MPZ miRNAs can encode one, two, three, four, five, six, seven or eight MP2 aspects, a separate rAAV is provided which encodes an RNAi-resistant replacement MPZ gene. In some aspects, the rAAV encodes the RNAi-resistant replacement MPZ gene in the same rAAV vector. In some aspects, the rAAV encoding one or more MPZ miRNAs can encode one, two, three or four MPZ miRNAs.

In some aspects, therefore, the viral vector is an AAV. The disclosure includes all types of AAV and is not limited to only the types of AAV described herein. Thus, such AAV include, but are not limited to, AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAV10 (i.e., an AAV containing AAV10 ITRs and AAV10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAV-anc80 (i.e., an AAV containing AAV-anc80 ITRs and AAV-anc80 capsid proteins), AAVrh.74 (i.e., an AAV containing AAVrh.74 ITRs and AAVrh.74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV-PHP.B, AAV-PHP.eB, AAV-PHP.S, AAVv66, AAV-F, or a pseudotyped AAV, such as AAV2/1, AAV2/8, or AAV2/9.

In various aspects, the viral vector is AAV9, AAV-PHP.eB, or AAV-F. In various aspects, the viral vector is AAV9 or AAV-F. AAV9 has become the most widely used vector for neurological indications with an established safety profile in the clinic. Intrathecal administration of AAV9 permits dissemination of transgenes throughout the nervous system and is currently approved by FDA for spinal muscular atrophy (SMA, NCT03381729), and in trials for the treatment of neuronal ceroid lipofuscinosis 3 (CLN3, NCT03770572), CLN6 (NCT02725580), and giant axonal neuropathy (GAN, NCT02362438). It is known that AAV9 can also target Schwann cells, a clear therapeutic target for CMT1B disease and other peripheral neuropathies. More importantly, AAV9 was reported to transduce Schwann cells in large animals and non-human primates (Bradbury et al., J Clin Invest, 2020, 130(9): 4906-4920; Gautier et al., Nat Commun, 2021, 12: 2356), indicating that it is a desirable viral vector for clinical applications requiring delivery of therapeutic genes into the human Schwann cells. AAV-F recently has been shown to be effective in the spinal cord (Beharry et al., Hum Gene Ther. 2022 January; 33 (1-2):61-75. doi: 10.1089/hum.2021.(26).

DNA plasmids of the disclosure comprise rAAV genomes of the disclosure. In some aspects, the DNA plasmids are transferred to cells permissible for infection with a helper virus of AAV (e.g., adenovirus, E1-deleted adenovirus or herpes virus) for assembly of the rAAV genome into infectious viral particles. Thus, in some aspects, the disclosure includes AAV vectors to deliver therapeutic agents into a cell. In some aspects, the cell is a neuronal cell. In some aspects, the neuronal cell is a Schwann cell.

Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, or AAVv66. In some aspects, AAV DNA in the rAAV genomes is from any AAV serotype for which a recombinant virus can be derived including, but not limited to, AAV serotypes AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant caps capsid protein containing a chimera of one or more of capsid proteins from AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol., 158: 97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production and use of self-complementary (sc) rAAV are specifically contemplated and exemplified.

The disclosure thus provides packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

Compositions comprising the nucleic acids and viral vectors of the disclosure are provided. Compositions comprising delivery vehicles (such as rAAV) described herein are provided. In various aspects, such compositions also comprise a pharmaceutically acceptable carrier. In some aspects, a pharmaceutically acceptable carrier is a diluent, excipient, or buffer. The compositions may also comprise other ingredients, such as adjuvants.

Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yields active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×10¹⁰ vg, 1×10¹¹ vg, 1×10¹² vg, 1×10¹³ vg, and 1×10¹⁴ vg, respectively).

Transduction of cells with rAAV of the disclosure results in sustained expression of MPZ miRNAs and RNAi-resistant replacement MPZ gene. The disclosure thus provides methods of administering/delivering rAAV which express MPZ miRNAs and an RNAi-resistant replacement MPZ gene to a subject. In some aspects, the subject is a mammal. In some aspects, the mammal is a human. These methods include transducing cells and tissues (including, but not limited to, peripheral motor neurons, sensory motor neurons, neurons, Schwann cells, and other tissues or organs, such as muscle, liver and brain) with one or more rAAV described herein. Transduction may be carried out with gene cassettes comprising cell-specific control elements.

The term “transduction” is used to refer to, as an example, the administration/delivery of MPZ miRNAs and RNAi-resistant replacement MPZ genes to a target cell either in vivo or in vitro, via a replication-deficient rAAV described herein resulting in the expression of MPZ miRNA and the RNAi-resistant replacement MPZ gene by the target cell.

Methods of transducing a target cell with a delivery vehicle (such as a nanoparticle, extracellular vesicle, exosome, or vector (e.g., rAAV)), in vivo or in vitro, are provided. The in vivo methods comprise the step of administering an effective dose, or effective multiple doses, of a composition comprising a delivery vehicle (such as rAAV) to an animal (including a human patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial o and/or that prolongs survival. Thus, methods are provided of administering an effective dose (or doses, administered essentially simultaneously or doses given at intervals) of rAAV described herein to a subject in need thereof.

Provided herein are medicaments and methods for treating, ameliorating, or preventing diseases associated with mutant MPZ gene or aberrant MPZ gene expression. MPZ gene encodes the MPZ protein, the major protein in the myelin sheath. MPZ is an essential protein in maintaining a healthy and efficient peripheral nervous system. Accumulation of defect protein in Schwann cells causes demyelination and cell death overtime. The pathological mechanisms of CMT1B disease can be mostly divided into two major groups: (1) toxic gain-of-function mutations that directly impact normal myelination, and (2) defective unfolded protein response (UPR) or endoplasmic reticulum (ER) stress responses. Both mechanisms of disease will finally lead to the accumulation of mutant myelin protein in Schwann Cells (SCs), reduced myelination, muscle weakness and atrophy, and loss of sensation in the lower legs and feet. For example, the R98C mutation causes an early onset, severe disease due to retaining mutated MPZ protein in ER and defective UPR. Patients carrying this mutation have very low or almost no myelin. These patients are classified as CMT1B but also as Dèjèrine-Sottas-Syndrome (DSS), another defining of very severe CMT1B or congenital hypomyelination. Thus, an example of a disease including for prevention, treatment or amelioration with methods of the disclosure is CMT1B disease. In families known to carry pathological MPZ mutations, methods for preventing are carried out in a subject before the onset of disease. In other subjects, the methods are carried out after diagnosis.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, one or more of the reduction or elimination of mutant MPZ mRNA or protein in affected tissues, MPZ gene knockdown, decreased demyelination or improved myelination. Others include, but are not limited to, decreased cell death.

In various aspects, quantitative RT-PCR (qRT-PCR) and western blot assays are used to detect expression of miMPZ and/or coMPZ. In some aspects, toluidine blue staining is carried out to investigate demyelination/remyelination and formation of onion bulbs in treated and untreated subjects. In some aspects, behavioral analyses, such as rotarod and foot grip tests, are performed to assess motor balance, coordination, and muscle strength. In some aspects, to determine the amount of Schwan cell death, TUNEL on sciatic nerves. In some aspects, tests are carried out to test the changes or stress in endoplasmic reticulum (ER) in muscle and unfolded protein response (UPR). In some aspects, these tests are carried out using a qRT-PCR assay. Skeletal muscle is a highly plastic tissue in the human body that undergoes extensive adaptation in response to environmental cues, such as physical activity, metabolic perturbation, and disease conditions. The ER plays a pivotal role in protein folding and calcium homeostasis in many mammalian cell types, including skeletal muscle. However, overload of misfolded or unfolded proteins in the ER lumen cause stress, which results in the activation of a signaling network called the UPR. The UPR is initiated by three ER transmembrane sensors: protein kinase R-like endoplasmic reticulum kinase, inositol-requiring protein 1α, and activating transcription factor 6. The UPR restores ER homeostasis through modulating the rate of protein synthesis and augmenting the gene expression of many ER chaperones and regulatory proteins. However, chronic heightened ER stress can also lead to many pathological consequences including cell death. Accumulating evidence suggests that ER stress-induced UPR pathways play pivotal roles in the regulation of skeletal muscle mass and metabolic function in multiple conditions. In some aspects a, nerve conduction velocity (NCV) test is used. During the test, the nerve is stimulated, usually with surface electrode patches attached to the skin. Two electrodes are placed on the skin over the nerve. One electrode stimulates the nerve with a very mild electrical impulse and the other electrode records it. The resulting electrical activity is recorded by another electrode. This is repeated for each nerve being tested and it provides a measurement of the speed of conduction of an electrical impulse through a nerve. Thus, in some aspects, the NCV test can determine nerve damage and destruction and/or improvement.

In the methods of the disclosure, expression of the mutant MPZ allele (or both mutant alleles) is inhibited by at least 10, at least 20, at least 30, at least 40, at least 50, at least 60, at least 70, at least 80, at least 90, at least 95, at least 98 percent, at least 99 percent, or 100 percent. In the methods, expression of the wild-type MPZ allele is inhibited by at least 10, at least 20, at least 30, at least 40, at least 50, at least 60, at least 70, at least 80, at least 90, at least 95, at least 98 percent, at least 99 percent, or 100 percent.

Combination therapies are also contemplated by the disclosure. In some aspects, the combination of two or more miRNA are used to achieve a greater silencing efficiency. In some aspects, the miRNA are used with other therapies for diseases associated with MPZ mutations. In some aspects, the miRNA described herein are used with other therapies designed to neutralize or decrease the expression of the mutant MPZ gene. In some aspects, such other therapies have been described herein. In some aspects, such other therapies are known to those of skill in the art. Combination includes both simultaneous treatment and sequential treatments. Combinations of methods described herein with standard medical treatments and supportive care are specifically contemplated, as are combinations with therapies.

Administration of an effective dose of a nucleic acid, nanoparticle, extracellular vesicle, exosome, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the miRNAs.

In particular, actual administration of delivery vehicle (such as rAAV) may be accomplished by using any physical method that will transport the delivery vehicle (such as rAAV) into a target cell of a subject. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the methods of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous med readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, suMPZ or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides kits for use in the treatment of a disease or disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, stabilizers, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Aspects and embodiments of the disclosure are illustrated by the following examples, which are not in any way meant to limit the scope of the invention.

### Example 1

### Materials and Methods

MicroRNA (miRNA) Design and Synthesis

MicroRNAs (miRNAs) specific for CMT1B Disease were designed and synthesized. The sequences of human MPZ cDNA (SEQ ID NO: 1; NM_000530.8) and its 3′UTR (SEQ ID NO: 2; NM_000530.8) are provided in Table 1. The sequence of partially codon-optimized MPZ cDNA (SEQ ID NO: 3) is provided in Table 1. Nucleotides 225-247 of SEQ ID NO: 3 (underlined in Table 1) contain the target site for miMPZ 225 and 226. Nucleotides 315-338 of SEQ ID NO: 3 (underlined in Table 1) contain the target site for miMPZ 315, 316, and 317. Nucleotides 718-744 of SEQ ID NO: 3 (underlined in Table 1) contain the target site for miMPZ 718, 719, 720, 721, 722, and 723. An entirely codon-optimized MPZ gene can be used to compare expression level of MPZ or to make new cassettes. Therefore, because more than one codon exists for each amino acid, other codon-optimized MPZ sequences are encompassed by the disclosure. The sequence of the MPZ protein sequence (SEQ ID NO: 4; NM_000530.8) is provided in Table 1. The sequence of the human MPZ promoter (SEQ ID NO: 5; ENSG00000158887) is provided in Table 1. The sequence of an MPZ expression cassette (SEQ ID NO: 6) made of 1200 bp of human MPZ promoter (ENSG00000158887), 63 bp human MPZ 5′UTR, and 747 bp of a partially codon optimized MPZ cDNA is provided in Table 1. SEQ ID NO: 6 also comprises an SV40 polyA signal and CMV amplicon sequences. The CMV amplicon is used in the construct for AAV titration using qRT-PCR or ddPCR assays. The lead miMPZ can be cloned into the NsiI or NotI sites located after the CMV amplicon. The entire sequence is cloned into an AAV pre-plasmid using two XbaI restriction enzyme sites at each end. The sequence was synthesized by Integrated DNA Technologies (IDT) (Coralville, IA). The order of sequences in coMPZ expression cassette: XBAI-stui-SalI-kasi-human (1200 bp)-NheI-human MPZ 5′UTR- partially codon optimized human mpz cds-SPEI-ndei-SV40 PA-paci-CMV AMPLICON FOR DRP-NSII-noti-XBAI. Alternately, the disclosure includes the use of various MPZ promoters or mini-MPZ promoters, including human, rat, or mouse promoters, in combination with universal enhancers, such as CMV-specific or Schwann-specific enhancers, such as PMP22.

Twelve synthetic miRNAs targeting MPZ (P0) gene (designated miMPZ) were designed and made. See Table 2. These miRNAs were designed to target a region on both the human and mouse MPZ gene where the MPZ gene sequence is conserved in both species. These are non-allele specific miMPZs, i.e., these miRNAs target both mutant and wild type MPZ and silence the overall MPZ expression in target cells. The miMPZ numbers signify the target sequence on cDNA and 3′UTR from which counting on the MPZ cDNA began. DNA encoding the miRNAs designated miMPZ_225, miMPZ_226, miMPZ_315, miMPZ_316, miMPZ_317, miMPZ_718, miMPZ_719, miMPZ_720, miMPZ_721, miMPZ_722, miMPZ_723, and miMPZ_1852 were generated by PCR using 12 pairs of primers as provided in Table 3.

PCR Cloning of miRNAs

One μg of each primer was added to a 1 cycle primer extension reaction: 95° C. for 5 min.; 94° C. for 2 min.; 52° C. for 1 min.; 68° C. for 15 min.; and then holding at 4° C. The PCR products were cleaned up with the Qiagen QIAquick PCR Purification kit before being digested overnight with XhoI and SpeI restriction enzymes. The digestion product was then run on a 1.5% TBE gel and the band excised and purified using the Qiagen QIAquick Gel Extraction Kit. The sequences of the each miRNA shown in FIG. 1A-1L.

The two PCR products were ligated overnight to a U6T6 vector (via XhoI and XbaI) that contains a mouse U6 promoter and an RNA polymerase III termination signal (six thymidine nucleotides). MiRNAs are cloned into XhoI and XbaI restriction sites located between the 3′ end of the U6 promoter and termination signal (SpeI site on the 3′ end of the DNA template for each miRNA has complementary cohesive ends with the XbaI site). The ligation product was transformed into chemically competent E. coli cells with a 42° C. heat shock and incubated at 37° C. shaking for 1 hour before being plated on kanamycin selection plates. The colonies were allowed to grow overnight at 37° C. The following day they were mini-prepped and sequenced for accuracy.

### Luciferase Assay

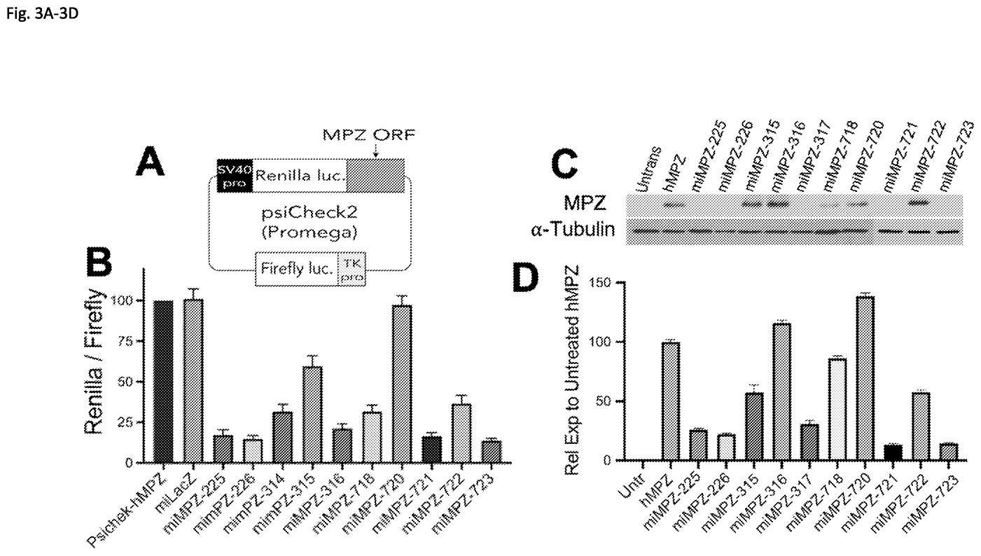

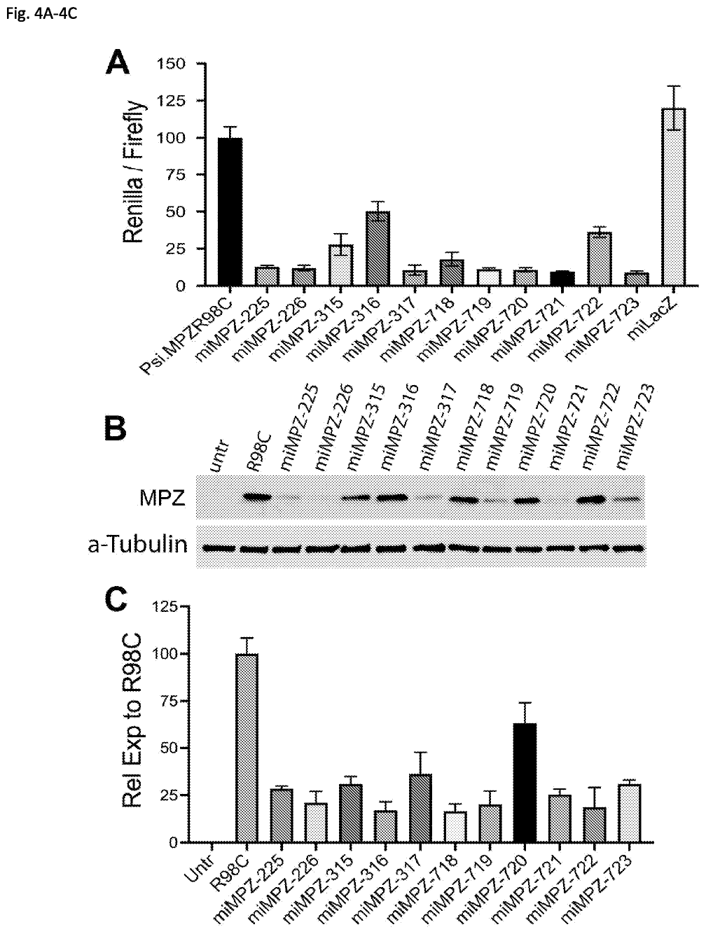

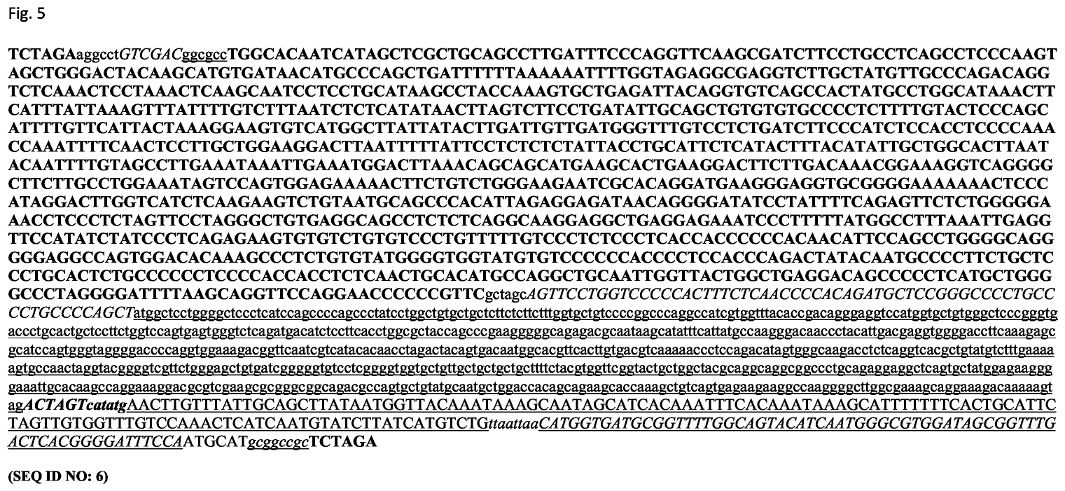

A luciferase assay was used to determine the expression level presence of the each miMPZ. 42,000 HEK293 cells were cultured in each well of a 96-well plate 16 hours before transfection. The next day, cells were transfected with 25 ng of a Renilla-firefly plasmid containing the MPZ target sequence (FIG. 3A) and 250 ng of each miMPZ-encoding plasmid using lipofectamine 2000. 24 hours later, a luciferase assay using Dual-Luciferase Reporter Assay kit (Promega, E1960) was carried out according to the manufacturer's protocol. The Renilla expression was divided by the firefly expression to calculate the relative expression. The relative expression was then normalized to the expression of cells that were transfected with a MPZ and U6T6 plasmids as negative control. Results are shown in FIGS. 3B-D. The MPZ targeting miRNA designated miMPZ-225, 226, 721, and 723 were the most effective at reducing luciferase protein expression in transfected cells in initial experiments.

### Western Blot

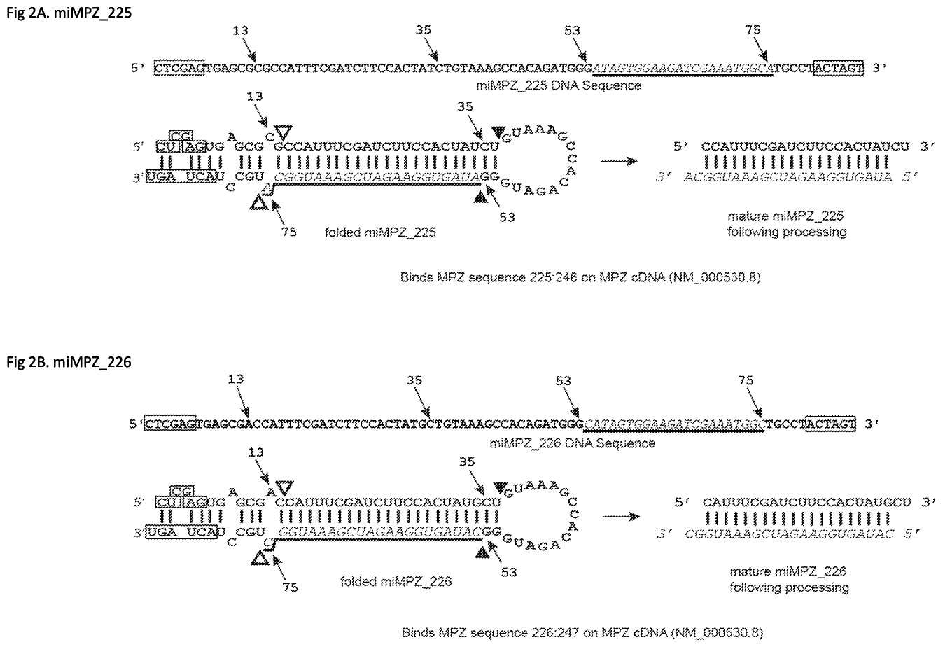

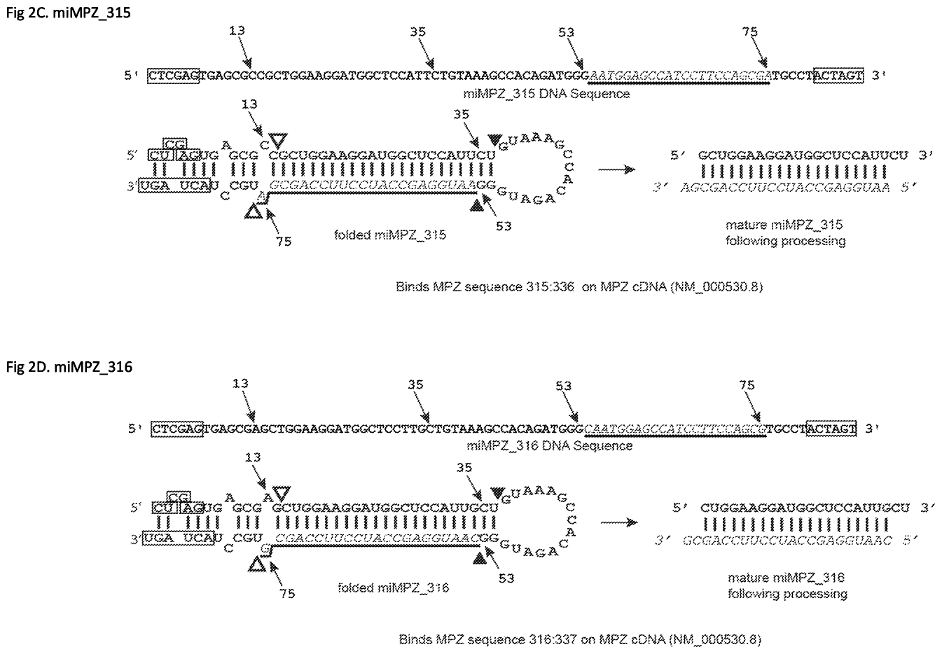

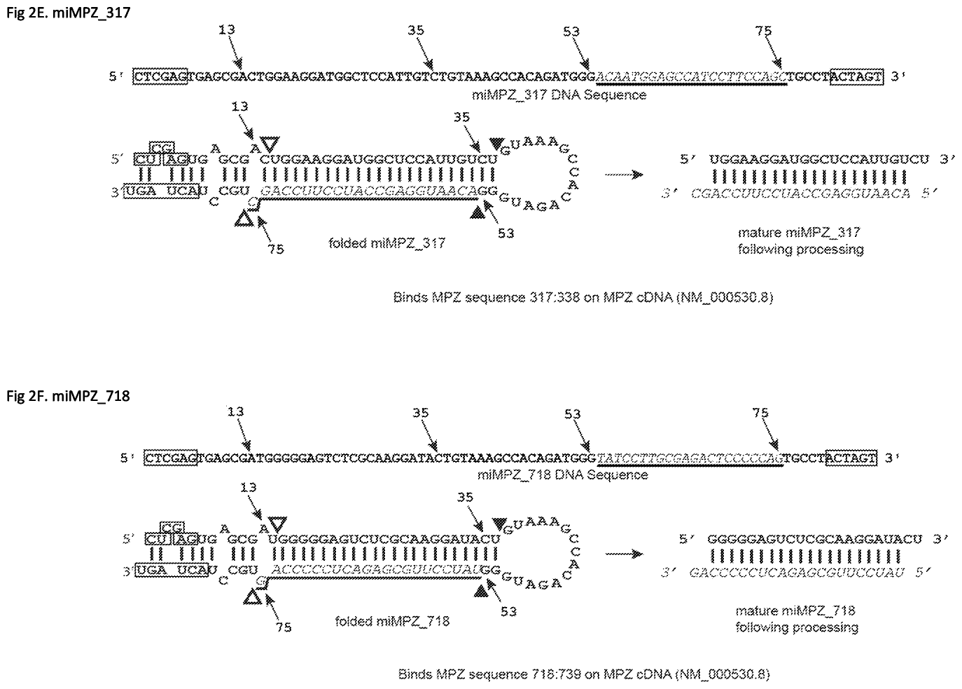

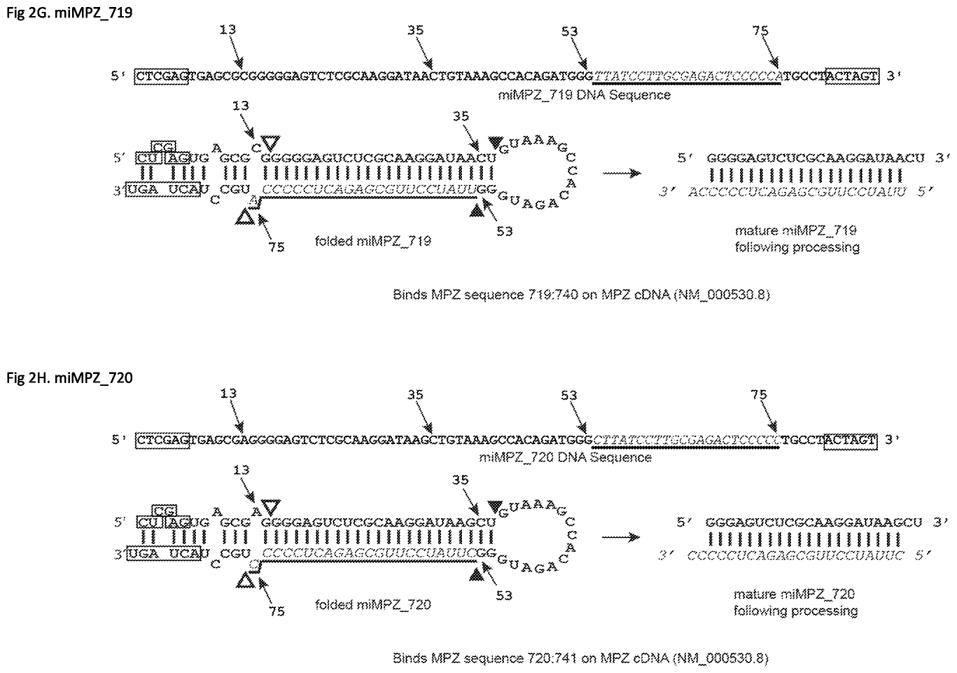

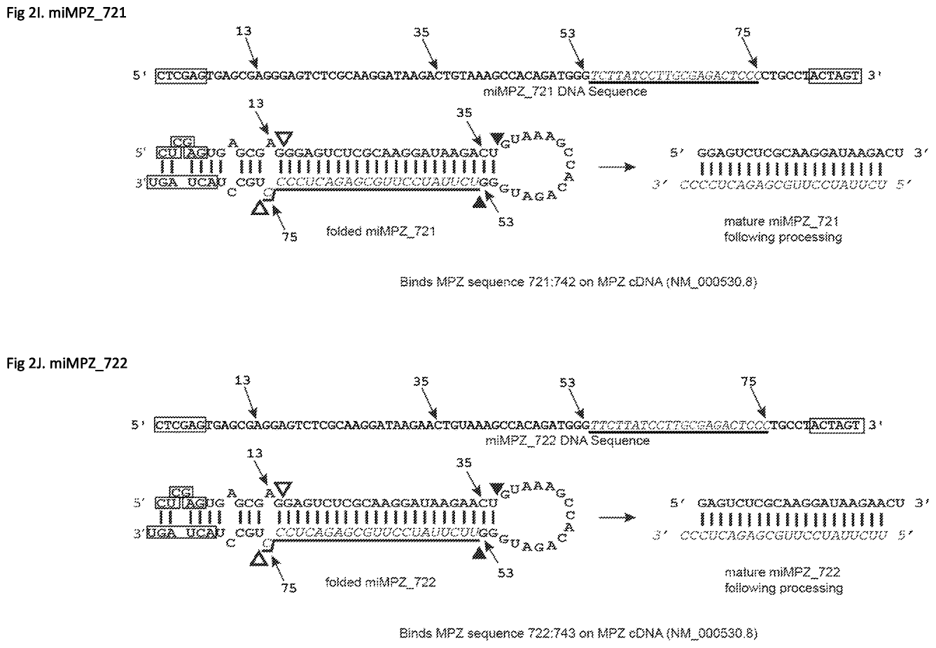

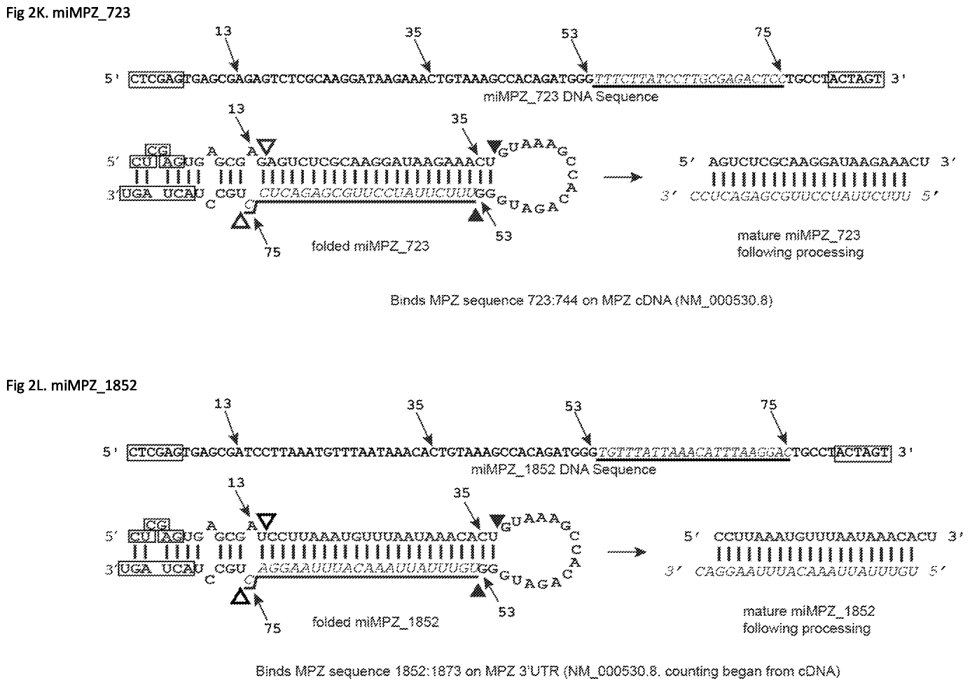

HEK293 cells (250,000 cells/well) were seeded in a 24-well plate 16 hrs before transfection. The next morning, they were co-transfected with 250 ng of MPZ and 1250 ng of miMPZ expression plasmids using Lipofectamine 2000 (Thermofisher, US). 24 hrs post-transfection, the cells were lysed in RIPA buffer (50 mM Tris, 150 mM NaCl, 0.1% SDS, 0.5% sodium deoxycholate, 1% Triton X 100) supplemented with a cocktail containing protease inhibitors. Protein concentration was determined by the DC protein assay kit (Bio-Rad Laboratories). 20 μg of each total protein sample were ran on 12% SDS-polyacrylamide gel. GE Healthcare Rainbow Molecular Weight Marker (Fisher scientific, USA) was used to determine the molecular weight of the protein bands. The proteins were transferred from SDS-PAGE gels onto PVDF membranes via semi-dried transfer method. The membrane was blocked in 5% non-fat milk, and then incubated with primary Rabbit anti-flag antibody (1:10,000, Abcam, ab1162), or rabbit polyclonal anti-α Tubulin antibodies (1:1,000; ab15246, Abcam, Cambridge, MA) overnight at 4° C. The next day following the washes, blots were then probed with horseradish-peroxidase-conjugated goat anti-rabbit secondary antibodies (1:100,000; Jackson ImmunoResearch, West Grove, PA) for 1 hour and 30 minutes at room temperature. Relative protein bands were developed on X-ray films after short incubation in Immobilon Chemiluminescent HRP Substrate (Millipore, Billerica, MA). The western blot results are shown in FIG. 2C. miMPZ 225, 226, 317, 721, and 723 were the most effective at knocking down MPZ protein expression.

qRT-PCR

MPZ and each miMPX plasmids, 250 and 1250 μg respectively, were co-transfected into HEK293 cells as explained in previous section for Western blot assay. Twenty-four hours later, the growth media was discarded and cells were PBS. Total RNA was extracted using Trizol (Trizol from Fisher, Waltham, MA) according to the manufactured protocol. The quality and quantity of isolated RNA was examined by Nanodrop, then was DNase-treated, and was used for RT-PCR using random hexamers (Applied Biosystems cDNA Archive Kit; Applied Biosystems, Foster City, CA). Subsequent cDNA samples were then used as a template for the Taqman Assay using predesigned human MPZ (Thermo Fisher, Hs01595271_g1) and human RPL13A control primer/probe sets (Thermofisher). Each sample were ran in triplicate. All data was normalized to MPZ only transfected cells. FIG. 3D shows the qRT-PCR results. The miMPZ_225, 721, and 723 were the most efficient miRNAs to reduce MPZ mRNA level.

MPZ Knockdown and Replacement Using rAAV Vectors as Gene Therapy for Treating CMT1B Disease

MPZ protein, also known as the major peripheral myelin protein or myelin protein zero (P0), is normally expressed by myelinating Schwann cells and makes more than 50% of the total PNS myelin protein. The miRNAs designed in this study target both mutant and wild-type MPZ genes. Therefore, to restore a healthy level of normal MPZ expression necessary for maintaining a healthy myelin sheath, an miMPZ-resistant MPZ gene is delivered via an AAV vector into the Schwann cells. To do that, the miMPZ target sites on human MPZ gene (NM_000530.8; SEQ ID NO: 1) were codon optimized so that miMPZ cannot attach to these regions. A codon-optimized MPZ cDNA (coMPZ) is provided as SEQ ID NO: 3. Both wild type and coMPZ produce the same healthy MPZ protein (SEQ ID NO: 4, NM_000530.8).

Finally, to do both knockdown and replacement, a gene expression cassette carrying “human MPZ promoter (hpMPZ)-coMPZ-U6-miMPZ” (SEQ ID NO: 6) was designed and made. To make this cassette, a cassette containing a human MPZ promoter (because MPZ is only expressed in Schwann cells, this promoter is one which is very suitable), human coMPZ, and restriction sites necessary for cloning miMPZ in this cassette were designed. The complete sequence and order of each part of the cassette is shown in FIG. 4. The expression of the MPZ protein is tested in vitro in HEK293 cells and then the ability of each miMPZ to target the MPZ target sequence is tested by Western blot and qRT-PCR. Later, after cloning a lead miMPZ into this plasmid, it will be packaged into an AAV vector using XbaI sites at each end of the sequence, for in vivo studies in CMT1B mice.

This cassette expresses two genes, one is the miMPZ expressed under a U6 promoter that provide silencing of endogenous (both mutant and wild-type) MPZ gene. The other gene is a healthy, codon-optimized MPZ (coMPZ) gene that is resistant to miMPZ-mediated gene silencing and expressed under a human Schwann-spec i.e., a 1200 bp endogenous human MPZ promoter (Seq ID 5, ENSG00000158887).

### Outcome Measures

The expression level of Mpz is investigated by qRT-PCR and western blot assays. Toluidine blue staining is carried out to investigate demyelination/remyelination and formation of onion bulbs. Behavioral analyses, such as rotarod and foot grip tests, are carried out to assess motor balance, coordination, and muscle strength. To determine the amount of Schwan cell death, TUNEL assays are performed on sciatic nerves. The changes in ER and UPR responses are determined using a qRT-PCR assay. To examine nerve histology, peripheral sciatic nerves of 2- or 6-month-old treated or control mice are dissected free, fixed, plastic embedded, sectioned at 1 μm and stained with toluidine blue. Axon number, axon size, g-ratios and myelin thickness, and onion bulb formations are measured in photomicrographs. Grip strength is tested by placing mice on a wire grid, which is then inverted, and the time to fall, up to one minute, is recorded. This test is used on mice three weeks of age and older, and is used longitudinally. Rotarod testing is used to test motor balance and coordination. Motor balance and coordination is determined using an accelerating rotarod apparatus. Mice are tested at 2- and 6-months of age. Training of animals consists of three trials per day with 15-min intervals for resting between trials, for 3 consecutive days. Mice are placed on the rod with the speed gradually increasing from 4 to 40 RPM. The trial ends when the mouse falls from the rod or when it remains on the rod for 600 s. Testing is performed on the 4th day using two different speeds, 20 and 32 RPM. Latency(s) to fall will be calculated for each speed.

The disclosure also provides nerve conduction velocity (NCV) testing. Mice are anesthetized with ketamine/xylazine, and the sciatic nerve is stimulated at the hip (sciatic notch) and ankle, and an EMG signal is recorded in the thenar muscles of the hind paw. The distance between stimulating electrodes divided by the difference in latency to the EMG determines the conduction velocity. EMG amplitude is determined. Such EMG amplitude can be integrated for the compound muscle action potential (iCMAP). In some aspects, a secondary EMG signal is recorded. The EMG signal results from the H-reflex, the monosynaptic reflex of the activated sensory neurons onto spinal motor neurons. Thus, muscle activity (EMG), sensory nerve conduction velocity, and synaptic connectivity in the spinal cord are measured.

### Example 2

### Efficacy of miRNA for Knocking Down MPZ Expression in Co-Transfected HEK293 Cells

miMPZs were generated by PCR and cloned in a plasmid under the control of the U6 promoter, which favors the expression of small hairpin loops by RNA polymerase III. Pre-screening of the miMPZ was carried out by co-transfecting miRNA and wild-type or mutant MPZ expression plasmids into HEK293 cells. In brief, the target sequence was incorporated into the 3′ untranslated region of Renilla luciferase in a Renilla/Firefly dual reporter system (FIG. 3A). The efficacy of target engagement and knockdown was determined by the luminescence ratio. FIGS. 3B and 4A show the luciferase assay results used to determine each miMPZ to target wild type and R98C mutant MPZ mRNA.

The use of miMPZ_225, 226, 317, 719, 721, and 723 provided an approximately >80% reduction in luciferase signal in both wild-type and mutant MPZ R98C. The same results were achieved using western blot and qRT-PCR from co-transfected HEK293 cells 24 hours post-transfection (FIGS. 3C and 4B). The same miMPZs caused a marked reduction of wildtype and mutant MPZ.

These results indicate that the miRNA were effective in knocking down MPZ expression in co-transfected HEK293 cells.

### Example 3

### Testing the Ability of miMPZ to Knock Down MPZ Expression in an Vitro Model of Dorsal Root Ganglia (DRG)/Schwann Cell Coculture from the R98C CMT1B Mouse Model

To evaluate the efficacy of MPZ R98C knockdown and replacement and investigate the re-myelination of axons in an in vitro model, a mouse dorsal root ganglion (DRT) and Schwann cell (DRG/Schwann) cell co-culture is used. This in vitro CMT1B model allows the study of CMT1B disease mechanisms and in vitro development of therapeutic constructs prior to in vivo experiments.

DRGs, isolated and purified from the embryonic spinal cord of R98C mice (Saporta et al., supra), provided by Dr. Michael Shy at the University of Iowa, are co-cultured with primary Schwann cells, as described by Florio et al. (J Neurosci. 2018, 38(18): 4275-4287). These cells are then transfected with each of the miMPZ described herein and any of MPZ or coMPZ described herein, or any of the expression cassettes described herein. In some aspects, each of the miMPZ and the coMPZ are provided in the same construct. In some aspects, each of the miMPZ and the coMPZ are provided in one or more constructs. Controls are treated with empty vector, coMPZ alone, miMPZ alone, or are untreated.

After a period of time, myelination is analyzed using various by various experimental methods, including immunostaining, Western blotting, qPCR, and qRT-PCR. qRT-PCR testing and Western blot assays are used to detect expression of miMPZ, mutant MPZ, and coMPZ. Toluidine blue staining is carried out to investigate demyelination/remyelination and formation of onion bulbs on the cells.

Improvement in myelination is obtained with treatment with miMPZ and coMPZ.

### Example 4

### miMPZ Knock Down and MPZ Replacement in the R98C Mouse Model of CMT1B

To validate and test the efficacy in vivo of knock down (AAV9-mediated MPZ gene silencing by miMPZ) and gene replacement by AAV9-coMPZ, knock down and gene replacement is carried out in a severe R98C CMT1B mouse model (Saporta et al., supra), provided by Dr. Michael Shy at the University of Iowa.

The AAV vector carrying any of the miMPZ and any of the coMPZ described herein is administered into the CSF of pre or post-symptomatic mice via either ICV or intrathecal injection or via direct injection into the sciatic nerve. Controls are treated with empty vector, coMPZ alone, miMPZ alone, or are untreated.

The efficacy of mutant MPZ knockdown and coMPZ replacement in improving myelination is investigated in the mice at several time points after administration using various methods including, but not limited to, immunostaining, Western blotting, qPCR, and qRT-PCR, as described herein.

qRT-PCR testing and Western blot assays are used to detect expression of miMPZ, mutant MPZ, and coMPZ. Toluidine blue staining is carried out to investigate demyelination/remyelination and formation of onion bulbs in treated and untreated mice. Behavioral analyses, such as rotarod and foot grip tests is performed to assess motor balance, coordination, and muscle strength. To determine the amount of Schwann cell death, TUNEL assays are carried out on sciatic nerves. The changes in ER and UPR responses are determined using qRT-PCR assay in treated and untreated mice. An NCV assay is used to investigate motor function improvement in treated mice compared to untreated animals.

Treatment with miMPZ knock down and coMPZ replacement in the R98C mouse model of CMT1B improves myelination, motor balance, motor function, coordination, and/or muscle strength in the mice.

### Example 5

### miMPZ Knock Down and MPZ Replacement in the S63del Mouse Model of CMT1B

To validate and test the efficacy in vivo of knock down (AAV9-mediated MPZ gene silencing by miMPZ) and gene replacement by AAV9-coMPZ, knock down and gene replacement is carried out in the S63del CMT1B mouse model (Wrabetz et al., (supra); Sidoli et al., J Neuroscience, 2016, 36(44):11350-61). This transgenic mouse model is proved by Dr. Lawrence Wrabetz and Dr. Laura Fetri at the University of Buffalo, NY.

The AAV vector carrying the miMPZ and coMPZ is administered into the CSF of pre or post-symptomatic mice via either ICV or intrathecal injection or via direct injection into the sciatic nerve. Controls are treated with empty vector, coMPZ alone, miMPZ alone, or are untreated.

The efficacy of mutant MPZ knockdown and coMPZ replacement in improving myelination is investigated in the mice at several time points after administration using various methods including, but not limited to, immunostaining, Western blotting, qPCR, and qRT-PCR, as described herein.

qRT-PCR testing and western blot assays are used to detect expression of miMPZ, mutant MPZ, and coMPZ. Toluidine blue staining is carried out to investigate demyelination/remyelination and formation of onion bulbs in treated and untreated mice. Behavioral analyses, such as rotarod and foot grip tests is performed to assess motor balance, coordination, and muscle strength. To determine the amount of Schwan cell death, TUNEL assays are carried out on sciatic nerves. The changes in ER and UPR responses are determined using qRT-PCR assay in treated and untreated mice. An NCV assay is used to investigate motor function improvement in treated mice compared to untreated animals.

Treatment with miMPZ knock down and coMPZ replacement in the S63del mouse model of CMT1B improves myelination, motor balance, motor function, coordination, and/or muscle strength in the mice.

### Example 6

### miMPZ Knock Down and MPZ Replacement or MPZ Replacement Alone in the Treatment of Human Patients with Mutations of MPZ

Mutations in MPZ cause CMT1B, and many of the >200 mutations cause neuropathy through a toxic gain of function by the mutant protein such as ER retention, activation of the Unfolded Protein Response (UPR) or disruption of myelin compaction. Patients have been identified who are heterozygous or homozygous for myelin P0 or Po gene (MPZ) mutations (Ikegami et al., Biochem. Biophys. Res. Commun. 1996; 222:107-110. Ikegami et al. (supra) identified a novel homozygous Phe 64 dele suggested that impairment of myelination is dosage-dependent. Other patients have been identified with mutations of MPZ resulting in haploinsufficiency of MPZ, which results in mild neurophathy, demonstration of axonal loss and various degrees of impairment to the lower and upper limbs (Howard et al., J. Peripher Nerv Syst. 2021; 26:177-83). Five patients had a heterozygous c.306 del; p.Asp104fs mutation and one patient had a c.204C>A; p.Tyr68Ter mutation of MPZ (Howard et al. (supra)). Howard et al. (supra) reported that in this group the patients' phenotype differs from that of the Mpz+/− mice because patients presented with predominantly large fiber sensory neuropathies, whereas the mice have motor greater than sensory nerve involvement. Howard et al. (supra) hypothesized that these patients may serve as candidates for therapeutic approaches which have been demonstrated to be effective in Mpz+/− mice and may also serve as models to compare other late onset phenotypes in patients with MPZ mutations.

To validate and test the efficacy of in vivo of knock down (AAV9-mediated MPZ gene silencing by miMPZ) and gene replacement by AAV9-coMPZ, knock down and gene replacement therapy is contemplated for treatment of human patients with both homozygous and heterozygous mutations of the MPZ gene. Additionally, MPZ gene replacement alone is contemplated as one means for treatment of these patients.

Thus, subject to regulatory approval, (i) an AAV vector carrying any one or more of the miMPZ and any of the coMPZ, (ii) an AAV vector carrying any of the coMPZ, or (iii) an AAV vector carrying an MPZ gene or a functional variant thereof is administered or delivered to these MPZ-deficient patients. In some aspects, the administration is into the CNS. In some aspects, the administration is via either ICV or intrathecal injection. In some aspects, the administration is by direct injection into the sciatic nerve.

Heterozygous MPZ Loss of Function (LOF) mutations causes milder neuropathy phenotypes whereas homozygous MPZ LOF mutations causes severe early onset CMT1B, also known as Dejerine-Sottas-Syndrome (DSS). MPZ gene replacement (i.e., delivery of either the codon-optimized MPZ gene or the wild-type MPZ gene or a functional MPZ gene variant) or MPZ knock down and gene replacement (i.e., delivery of miMPZ with coMPZ) are therapeutic options for both heterozygous and homozygous LOF mutation patients.

Both gene replacement alone, and knockdown and replacement therapy methods are useful in the treatment of these patients. Likewise, therefore, both wild-type and codon-optimized MPZ could be used in treatment.

While the present disclosure has been described in terms of specific embodiments, it is understood that variations and modifications will occur to those skilled in the art. Accordingly, only such limitations as appear in the claims should disclosure.

All documents referred to in this application are hereby incorporated by reference in their entirety.

We claim:

1. A nucleic acid comprising
a) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
b) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
c) a polynucleotide sequence comprising or encoding an MPZ microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42;
d) a polynucleotide sequence that specifically hybridizes to a target nucleotide sequence on the MPZ gene, wherein the target nucleotide sequence is set forth in any one of SEQ ID NOs: 43-54; or
e) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

2. A nucleic acid comprising
a) i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18; or
ii) a polynucleotide sequence that specifically hybrid nucleotide sequence on the MPZ gene, wherein the target nucleotide sequence is set forth in any one of SEQ ID NOs: 43-54; and
b) i) a polynucleotide sequence encoding a human MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 1;
ii) a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 3; or
iii) a polynucleotide sequence encoding an MPZ polypeptide sequence comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 4.

3. The nucleic acid of claim 2 further comprising a promoter or multiple promoters.

4. The nucleic acid of claim 3, wherein the promoter is a U6 promoter, a U7 promoter, an H19 promoter, a neuron-specific promoter, or a Schwann cell-specific promoter.

5. The nucleic acid of claim 3, wherein the Schwann cell-specific promoter is an MPZ promoter or a mini-MPZ promoter.

6. The nucleic acid of claim 5, wherein the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5.

7. A nanoparticle, extracellular vesicle, exosome, or vector comprising the nucleic acid of any one of claims 1-6 or a combination of any one or more thereof.

8. The vector of claim 7, wherein the vector is a viral vector.

9. The viral vector of claim 8, wherein the viral vector is an virus (AAV), adenovirus, lentivirus, retrovirus, poxvirus, baculovirus, herpes simplex virus, vaccinia virus, or a synthetic virus.

10. The viral vector of claim 8 or 9, wherein the viral vector is an AAV.

11. The viral vector of claim 10, wherein the AAV lacks rep and cap genes.

12. The viral vector of claim 10 or 11, wherein the AAV is a recombinant AAV (rAAV), a self-complementary recombinant AAV (scAAV), or a single-stranded recombinant AAV (ssAAV).

13. The AAV of any one of claims 10-12, wherein the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F.

14. The AAV of any one of claims 10-13, wherein the AAV is AAV9, AAVrh.10, AAV-PHP.eB, AAVv66, or AAV-F.

15. A composition comprising
(a) the nucleic acid of any one of claims 1-6;
(b) the nanoparticle, extracellular vesicle, exosome, or vector of claim 7; or
(c) the viral vector of any one of claims 8-14; and
a pharmaceutically acceptable carrier.

16. A method of reducing the expression of a mutant myelin protein zero (MPZ) gene in a cell comprising contacting the cell with:
(a) the nucleic acid of any one of claims 1-6;
(b) the nanoparticle, extracellular vesicle, exosome, or vector of claim 7; or
(c) the viral vector of any one of claims 8-14; or
(d) the composition of claim 15.

17. The method of claim 16, wherein the cell is a neuronal c

18. The method of claim 17, wherein the neuronal cell is a Schwann cell.

19. The method of any one of claims 17 and 18, wherein the cell is a human cell.

20. The method of claim 19, wherein the cell is in a human subject.

21. The method of claim 20, wherein the subject suffers from a hypomyelination disease or a Charcot-Marie-Tooth disease.

22. The method of claim 21, wherein the disease is CMT (DI-CMT), CMT type 1B (CMT1B), CMT type 2I (CMT2I), CMT type 2J (CMT2J), or Dejerine-Sottas Syndrome (DSS or CMT type 3) disease.

23. A method of treating a subject comprising a mutant myelin protein zero (MPZ) gene, the method comprising administering to the subject an effective amount of:
(a) the nucleic acid of any one of claims 1-6;
(b) the nanoparticle, extracellular vesicle, exosome, or vector of claim 7; or
(c) the viral vector of any one of claims 8-14; or
(d) the composition of claim 15.

24. The method of claim 23, wherein the subject is a human subject.

25. The method of claim 24, wherein the subject suffers from a hypomyelination disease or a Charcot-Marie-Tooth disease.

26. The method of claim 25, wherein the disease is CMT (DI-CMT), CMT type 1B (CMT1B), CMT type 2I (CMT2I), CMT type 2J (CMT2J), or Dejerine-Sottas Syndrome (DSS or CMT type 3) disease.

27. The method of claim 16 or 23, wherein the nucleic acid is any one of claims 2-6.

28. The method of claim 27, wherein the nucleic acid is present in a composition and/or in a nanoparticle, extracellular vesicle, exosome, or vector.

29. The method of claim 27 or 28, wherein the vector is a vir

30. The method of claim 29, wherein the viral vector is AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F.

31. The method of claim 29, wherein the AAV is AAV9, AAVrh.10, AAV-PHP.eB, AAVv66, or AAV-F.

32. Use of
(a) the nucleic acid of any one of claims 1-6;
(b) the nanoparticle, extracellular vesicle, exosome, or vector of claim 7; or
(c) the viral vector of any one of claims 8-14; or
(d) the composition of claim 15 for the preparation of a medicament for reducing expression of a mutant myelin protein zero (MPZ) gene in a cell.

33. The use of claim 32, wherein the cell is in a human subject.

34. Use of
(a) the nucleic acid of any one of claims 1-6;
(b) the nanoparticle, extracellular vesicle, exosome, or vector of claim 7; or
(c) the viral vector of any one of claims 8-14; or
(d) the composition of claim 15
in treating a subject comprising a mutant myelin protein zero (MPZ) gene.

35. The use of claim 34, wherein the subject is a human subject.

36. The use of claim 33 or 35, wherein the subject suffers from a hypomyelination disease or a Charcot-Marie-Tooth disease.

37. The use of claim 36, wherein the disease is CMT (DI-CMT (CMT1B), CMT type 2I (CMT2I), CMT type 2J (CMT2J), or Dejerine-Sottas Syndrome (DSS or CMT type 3) disease.

38. The
(a) nucleic acid of any one of claims 1-6;
(b) nanoparticle, extracellular vesicle, exosome, or vector of claim 7;
(c) viral vector of any one of claims 8-14;
(d) composition of claim 15;
(e) method of any one of claims 16-31; or
(f) use of any one of claims 32-37,
wherein the nucleic acid, nanoparticle, extracellular vesicle, exosome, vector, viral vector, composition, or medicament is formulated for intramuscular injection, oral administration, subcutaneous, intradermal, or transdermal transport, injection into the blood stream, or for aerosol administration.

39. A method of reducing expression of a mutant myelin protein zero (MPZ) gene in a cell and expressing a functional MPZ protein in a cell, the method comprising delivering to the cell an effective amount of
(a) a nucleic acid comprising one or more of
(i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
(ii) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
(iii) a polynucleotide sequence comprising or encoding microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42; and/or
(iv) a polynucleotide sequence that specifically hybridizes to a target nucleotide sequence on the MPZ gene, wherein the target nucleotide sequence is set forth in any one of SEQ ID NOs: 43-54; and
(b) a nucleic acid comprising a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 3 or 6.

40. The method of claim 39, wherein any one or more of the nucleic acids further comprises a promoter or multiple promoters.

41. The method of claim 40, wherein the promoter is a U6 promoter, a U7 promoter, an H19 promoter, a neuron-specific promoter, or a Schwann cell-specific promoter.

42. The method of claim 41, wherein the Schwann cell-specific promoter is an MPZ promoter or a mini-MPZ promoter.

43. The method of claim 42, wherein the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5.

44. The method of any one of claims 39-43, wherein the cell is a neuronal cell.

45. The method of claim 44, wherein the neuronal cell is a Schwann cell.

46. The method of any one of claims 39-45, wherein the cell is a human cell.

47. The method of any one of claims 39-46, wherein the cell is in a human subject.

48. The method of claim 47, wherein the subject suffers from disease or a Charcot-Marie-Tooth disease.

49. The method of claim 48, wherein the disease is CMT (DI-CMT), CMT type 1B (CMT1B), CMT type 2I (CMT2I), CMT type 2J (CMT2J), or Dejerine-Sottas Syndrome (DSS or CMT type 3) disease.

50. The method of any one of claims 39-49, wherein the nucleic acid is delivered to the cell in a nanoparticle, an extracellular vesicle, an exosome, or a vector, or a combination of any one or more thereof.

51. The method of claim 50, wherein the vector is a viral vector.

52. The method of claim 51, wherein the viral vector is an adeno-associated virus (AAV), adenovirus, lentivirus, retrovirus, poxvirus, baculovirus, herpes simplex virus, vaccinia virus, or a synthetic virus.

53. The method of claim 51 or 52, wherein the viral vector is an AAV.

54. The method of claim 53, wherein the AAV lacks rep and cap genes.

55. The method of claim 53 or 54, wherein the AAV is a recombinant AAV (rAAV), a self-complementary recombinant AAV (scAAV), or a single-stranded recombinant AAV (ssAAV).

56. The method of any one of claims 53-55, wherein the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F.

57. The method of claim 56, wherein the AAV is AAV9, AAVrh.10, AAV-PHP.eB, AAVv66, or AAV-F.

58. The method of any one of claims 50-57, wherein
the nucleic acid comprising the polynucleotide sequence comprising or encoding the MPZ miRNA and/or the polynucleotide sequence that specifically hybridizes to a target nucleotide sequence on the MPZ gene, and
the nucleic acid comprising the polynucleotide sequence encoding optimized MPZ DNA
are delivered to the cell at the same time.

59. The method of claim 58, wherein the nucleic acids are delivered to the cell in the same vector.

60. The method of any one of claims 50-57, wherein
the nucleic acid comprising the polynucleotide sequence comprising or encoding the MPZ miRNA and/or the polynucleotide sequence that specifically hybridizes to a target nucleotide sequence on the MPZ gene, and
the nucleic acid comprising the polynucleotide sequence encoding the codon-optimized MPZ DNA
are delivered to the cell at different times.

61. A method of treating a subject suffering from aberrant expression of a mutant myelin protein zero (MPZ) gene, the method comprising reducing expression of the mutant MPZ gene and expressing functional MPZ protein in the subject, the method comprising delivering to the subject an effective amount of
(a) a nucleic acid comprising one or more of
(i) a polynucleotide sequence encoding a myelin protein zero (MPZ) microRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 7-18;
(ii) a polynucleotide sequence comprising an MPZ microRNA, wherein the MPZ microRNA comprises the nucleotide sequence set forth in any one of SEQ ID NOs: 19-30 or a variant thereof comprising at least about 90% identity to the nucleotide sequence set forth in any one of SEQ ID NOs: 19-30;
(iii) a polynucleotide sequence comprising or encoding microRNA, wherein the MPZ microRNA comprises the nucleotide sequence set forth in any one of SEQ ID NOs: 31-42 or a variant thereof comprising at least about 90% identity to the nucleotide sequence set forth in any one of SEQ ID NOs: 31-42; and/or
(iv) a polynucleotide sequence that specifically hybridizes to a target nucleotide sequence on the MPZ gene, wherein the target nucleotide sequence is set forth in any one of SEQ ID NOs: 43-54; and
(b) a nucleic acid comprising a polynucleotide sequence encoding a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 3 or 6.

62. The method of claim 61, wherein any one or more of the nucleic acids further comprises a promoter or multiple promoters.

63. The method of claim 62, wherein the promoter is a U6 promoter, a U7 promoter, an H19 promoter, a neuron-specific promoter, or a Schwann cell-specific promoter.

64. The method of claim 63, wherein the Schwann cell-specific promoter is an MPZ promoter or a mini-MPZ promoter.

65. The method of claim 64, wherein the MPZ promoter comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in SEQ ID NO: 5.

66. The method of any one of claims 61-65, wherein the subject is a human.

67. The method of claim 66, wherein the subject suffers from a hypomyelination disease or a Charcot-Marie-Tooth disease.

68. The method of claim 67, wherein the disease is CMT (DI-CMT), CMT type 1B (CMT1B), CMT type 2I (CMT2I), CMT type 2J (CMT2J), or Dejerine-Sottas Syndrome (DSS or CMT type 3) disease.

69. The method of any one of claims 61-68, wherein the nuc to the subject in a nanoparticle, an extracellular vesicle, an exosome, or a vector, or in a combination of any one or more thereof.

70. The method of claim 69, wherein the vector is a viral vector.

71. The method of claim 70, wherein the viral vector is an adeno-associated virus (AAV), adenovirus, lentivirus, retrovirus, poxvirus, baculovirus, herpes simplex virus, vaccinia virus, or a synthetic virus.

72. The method of claim 70 or 71, wherein the viral vector is an AAV.

73. The method of claim 72, wherein the AAV lacks rep and cap genes.

74. The method of claim 72 or 73, wherein the AAV is a recombinant AAV (rAAV), a self-complementary recombinant AAV (scAAV), or a single-stranded recombinant AAV (ssAAV).

75. The method of any one of claims 72-74, wherein the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAV-PHP.B, AAV-PHP.eB, AAV-PHP.S, AAVv66, or AAV-F.

76. The method of claim 75, wherein the AAV is AAV9, AAVrh.10, AAV-PHP.eB, AAVv66, or AAV-F.

77. The method of any one of claims 61-76, wherein
the nucleic acid comprising the polynucleotide sequence comprising or encoding the MPZ miRNA and/or the polynucleotide sequence that specifically hybridizes to a target nucleotide sequence on the MPZ gene, and
the nucleic acid comprising the polynucleotide sequence encoding the codon-optimized MPZ DNA
are delivered to the subject at the same time.

78. The method of claim 77, wherein the nucleic acids are delivered to the subject in the same vector.

79. The method of any one of claims 61-76, wherein
the nucleic acid comprising the polynucleotide sequence comprising or encoding the MPZ miRNA and/or the polynucleotide sequence that specifically hybridizes to a target nucleotide sequence on the MPZ gene, and
the nucleic acid comprising the polynucleotide sequence encoding the codon-optimized MPZ DNA
are delivered to the subject at different times.

80. A method of treating a subject suffering from aberrant expression of a mutant myelin protein zero (MPZ) gene, the method comprising expressing functional MPZ protein in the subject by delivering to the subject an effective amount of a nucleic acid comprising a polynucleotide sequence encoding an MPZ DNA or a codon-optimized MPZ DNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1, 3, and 6.


SEQUENCE LISTING (84 sequences)

SEQ ID NO: 1 - DNA, 747 bp, Homo sapiens
  source 1..747: mol_type = other DNA
atggctcctggggctccctcatccagccccagccctatcctggctgtgctgctcttctcttctttggtgctgtccccggcccaggccatcgtggtttacaccgacagggaggtccatggtgctgtgggctcccgggtgaccctgcactgctccttctggtccagtgagtgggtctcagatgacatctccttcacctggcgctaccagcccgaagggggcagagatgccatttcgatcttccactatgccaagggacaaccctacattgacgaggtggggaccttcaaagagcgcatccagtgggtaggggaccctcgctggaaggatggctccattgtcatacacaacctagactacagtgacaatggcacgttcacttgtgacgtcaaaaaccctccagacatagtgggcaagacctctcaggtcacgctgtatgtctttgaaaaagtgccaactaggtacggggtcgttctgggagctgtgatcgggggtgtcctcggggtggtgctgttgctgctgctgcttttctacgtggttcggtactgctggctacgcaggcaggcggccctgcagaggaggctcagtgctatggagaaggggaaattgcacaagccaggaaaggacgcgtcgaagcgcgggcggcagacgccagtgctgtatgcaatgctggaccacagcagaagcaccaaagctgtcagtgagaagaaggccaaggggctgggggagtctcgcaaggataagaaatag

SEQ ID NO: 2 - DNA, 1141 bp, Homo sapiens
  source 1..1141: mol_type = other DNA
cggttagcgggccgggcgggggatcgggggttaggggtggagtccgccaaaggcccaaaggtgatggtcatcgagatggagctacgaaaggatgagcagagcccggagctccggcctgctgtcaagtcccccagcagaaccagcctcaaaaacgccctcaagaacatgatgggcctgaactcggacaagtgatcgccacccccccaccccaggccctgccagagcagggggacctaggctcctcttacccccgtctaggtgctttccctctttgctcccccgccctgccctgccctcacctccctttgagatgtaagtttcattccagaattcattccccaggcaattgtattctcccccaccttcacccctggctttctgggagcccaggagctaatcctacccctcacctgccccgggggctgtgtgtttggtgcctgtccacctgagcactgagaagaaagggactttgataccctctgcctcaagtccaggccacctggcattcccatctcctgcatcccccagcctgtccccctggctgtttcctctcccgtccctccctcccctctaccaggtggcccagctccatactctgtccccccagctaatacccagagcacccagatcagactctccttcagggtttatttaggttattattttttattttttaatccattctttgtttgtttacctgtgctcatcctctgcccttacacccatgactgaggaccaatgacgtcatgtggcttttgcaattcacgccccccttaagtccttaatgaagagccagcccaagtagaggggcccctgatcctcacacttcagtatagcattggttccccctgaccactttggagcactgttctgggactccaggtcttgaggagagagacagagagagagaatggatcctcataggtcagggagtgggggagggggcaaatgagccttaagaaatggtttttaaacaaccaaacaaaaagcaggaaaaacaaatgggaaatgggggggcgggggggaggaagaggctgcactgcagccacaggggattcttaggatttttctacattctgtatatttcttctcaaacctccaaatgtccttaaatgtttaataaacactgacatttccagaa

SEQ ID NO: 3 - DNA, 747 bp, synthetic construct
  misc_feature 1..747: note = Partially codon optimized MPZ cDNA
  misc_feature 225..247: note = target site for miMPZ 225 and 226
  misc_feature 315..338: note = target sites for miMPZ 315, 316, 317
  misc_feature 718..744: note = target site for miMPZ 718, 719, 720, 721, 722, 723
  source 1..747: mol_type = other DNA
atggctcctggggctccctcatccagccccagccctatcctggctgtgctgctcttctcttctttggtgctgtccccggcccaggccatcgtggtttacaccgacagggaggtccatggtgctgtgggctcccgggtgaccctgcactgctccttctggtccagtgagtgggtctcagatgacatctccttcacctggcgctaccagcccgaagggggcagagacgcaataagcatatttcattatgccaagggacaaccctacattgacgaggtggggaccttcaaagagcgcatccagtgggtaggggaccccaggtggaaagacggttcaatcgtcatacacaacctagactacagtgacaatggcacgttcacttgtgacgtcaaaaaccctccagacatagtgggcaagacctctcaggtcacgctgtatgtctttgaaaaagtgccaactaggtacggggtcgttctgggagctgtgatcgggggtgtcctcggggtggtgctgttgctgctgctgcttttctacgtggttcggtactgctggctacgcaggcaggcggccctgcagaggaggctcagtgctatggagaaggggaaattgcacaagccaggaaaggacgcgtcgaagcgcgggcggcagacgccagtgctgtatgcaatgctggaccacagcagaagcaccaaagctgtcagtgagaagaaggccaaggggcttggcgaaagcaggaaagacaaaaagtag

SEQ ID NO: 4 - protein, 248 aa, Homo sapiens
MAPGAPSSSPSPILAVLLFSSLVLSPAQAIVVYTDREVHGAVGSRVTLHCSFWSSEWVSDDISFTWRYQPEGGRDAISIFHYAKGQPYIDEVGTFKERIQWVGDPRWKDGSIVIHNLDYSDNGTFTCDVKNPPDIVGKTSQVTLYVFEKVPTRYGVVLGAVIGGVLGVVLLLLLLFYVVRYCWLRRQAALQRRLSAMEKGKLHKPGKDASKRGRQTPVLYAMLDHSRSTKAVSEKKAKGLGESRKDKK

SEQ ID NO: 5 - DNA, 1200 bp, Homo sapiens
  source 1..1200: mol_type = other DNA
tggcacaatcatagctcgctgcagccttgatttcccaggttcaagcgatcttcctgcctcagcctcccaagtagctgggactacaagcatgtgataacatgcccagctgattttttaaaaaattttggtagaggcgaggtcttgctatgttgcccagacaggtctcaaactcctaaactcaagcaatcctcctgcataagcctaccaaagtgctgagattacaggtgtcagccactatgcctggcataaacttcatttattaaagtttattttgtctttaatctctcatataacttagtcttcctgatattgcagctgtgtgtgcccctcttttgtactcccagcattttgttcattactaaaggaagtgtcatggcttattatacttgattgttgatgggtttgtcctctgatcttcccatctccacctccccaaaccaaattttcaactccttgctggaaggacttaatttttattcctctctctattacctgcattctcatactttacatattgctggcacttaatacaattttgtagccttgaaataaattgaaatggacttaaacagcagcatgaagcactgaaggacttcttgacaaacggaaaggtcaggggcttcttgcctggaaatagtccagtggagaaaaacttctgtctgggaagaatcgcacaggatgaagggaggtgcggggaaaaaaactcccataggacttggtcatctcaagaagtctgtaatgcagcccacattagaggagataacaggggatatcctattttcagagttctctgggggaaacctccctctagttcctagggctgtgaggcagcctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttc

SEQ ID NO: 6 - DNA, 2278 bp, synthetic construct
  misc_feature 1..2278: note = MPZ expression cassette
  source 1..2278: mol_type = other DNA
tctagaaggcctgtcgacggcgcctggcacaatcatagctcgctgcagccttgatttcccaggttcaagcgatcttcctgcctcagcctcccaagtagctgggactacaagcatgtgataacatgcccagctgattttttaaaaaattttggtagaggcgaggtcttgctatgttgcccagacaggtctcaaactcctaaactcaagcaatcctcctgcataagcctaccaaagtgctgagattacaggtgtcagccactatgcctggcataaacttcatttattaaagtttattttgtctttaatctctcatataacttagtcttcctgatattgcagctgtgtgtgcccctcttttgtactcccagcattttgttcattactaaaggaagtgtcatggcttattatacttgattgttgatgggtttgtcctctgatcttcccatctccacctccccaaaccaaattttcaactccttgctggaaggacttaatttttattcctctctctattacctgcattctcatactttacatattgctggcacttaatacaattttgtagccttgaaataaattgaaatggacttaaacagcagcatgaagcactgaaggacttcttgacaaacggaaaggtcaggggcttcttgcctggaaatagtccagtggagaaaaacttctgtctgggaagaatcgcacaggatgaagggaggtgcggggaaaaaaactcccataggacttggtcatctcaagaagtctgtaatgcagcccacattagaggagataacaggggatatcctattttcagagttctctgggggaaacctccctctagttcctagggctgtgaggcagcctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcgctagcagttcctggtcccccactttctcaaccccacagatgctccgggcccctgcccctgccccagctatggctcctggggctccctcatccagccccagccctatcctggctgtgctgctcttctcttctttggtgctgtccccggcccaggccatcgtggtttacaccgacagggaggtccatggtgctgtgggctcccgggtgaccctgcactgctccttctggtccagtgagtgggtctcagatgacatctccttcacctggcgctaccagcccgaagggggcagagacgcaataagcatatttcattatgccaagggacaaccctacattgacgaggtggggaccttcaaagagcgcatccagtgggtaggggaccccaggtggaaagacggttcaatcgtcatacacaacctagactacagtgacaatggcacgttcacttgtgacgtcaaaaaccctccagacatagtgggcaagacctctcaggtcacgctgtatgtctttgaaaaagtgccaactaggtacggggtcgttctgggagctgtgatcgggggtgtcctcggggtggtgctgttgctgctgctgcttttctacgtggttcggtactgctggctacgcaggcaggcggccctgcagaggaggctcagtgctatggagaaggggaaattgcacaagccaggaaaggacgcgtcgaagcgcgggcggcagacgccagtgctgtatgcaatgctggaccacagcagaagcaccaaagctgtcagtgagaagaaggccaaggggcttggcgaaagcaggaaagacaaaaagtagactagtcatatgaacttgtttattgcagcttataatggttacaaataaagcaatagcatcacaaatttcacaaataaagcatttttttcactgcattctagttgtggtttgtccaaactcatcaatgtatcttatcatgtctgttaattaacatggtgatgcggttttggcagtacatcaatgggcgtggatagcggtttgactcacggggatttccaatgcatgcggccgctctaga

SEQ ID NO: 7 - DNA, 86 bp, synthetic construct
  misc_feature 1..86: note = Synthetic
  source 1..86: mol_type = other DNA
ctcgagtgagcgcgccatttcgatcttccactatctgtaaagccacagatgggatagtggaagatcgaaatggcatgcctactagt

SEQ ID NO: 8 - DNA, 86 bp, synthetic construct
  misc_feature 1..86: note = Synthetic
  source 1..86: mol_type = other DNA
ctcgagtgagcgaccatttcgatcttccactatgctgtaaagccacagatgggcatagtggaagatcgaaatggctgcctactagt

SEQ ID NO: 9 - DNA, 86 bp, synthetic construct
  misc_feature 1..86: note = Synthetic
  source 1..86: mol_type = other DNA
ctcgagtgagcgccgctggaaggatggctccattctgtaaagccacagatgggaatggagccatccttccagcgatgcctactagt

SEQ ID NO: 10 - DNA, 85 bp, synthetic construct
  misc_feature 1..85: note = Synthetic
  source 1..85: mol_type = other DNA
ctcgagtgagcgagctggaaggatggctccttgctgtaaagccacagatgggcaatggagccatccttccagcgtgcctactagt

SEQ ID NO: 11 - DNA, 86 bp, synthetic construct
  misc_feature 1..86: note = Synthetic
  source 1..86: mol_type = other DNA
ctcgagtgagcgactggaaggatggctccattgtctgtaaagccacagatgggacaatggagccatccttccagctgcctactagt

SEQ ID NO: 12 - DNA, 86 bp, synthetic construct
  misc_feature 1..86: note = Synthetic
  source 1..86: mol_type = other DNA
ctcgagtgagcgatgggggagtctcgcaaggatactgtaaagccacagatgggtatccttgcgagactcccccagtgcctactagt

SEQ ID NO: 13 - DNA, 86 bp, synthetic construct
  misc_feature 1..86: note = Synthetic
  source 1..86: mol_type = other DNA
ctcgagtgagcgcgggggagtctcgcaaggataactgtaaagccacagatgggttatccttgcgagactcccccatgcctactagt

SEQ ID NO: 14 - DNA, 86 bp, synthetic construct
  misc_feature 1..86: note = Synthetic
  source 1..86: mol_type = other DNA
ctcgagtgagcgaggggagtctcgcaaggataagctgtaaagccacagatgggcttatccttgcgagactccccctgcctactagt

SEQ ID NO: 15 - DNA, 86 bp, synthetic construct
  misc_feature 1..86: note = Synthetic
  source 1..86: mol_type = other DNA
ctcgagtgagcgagggagtctcgcaaggataagactgtaaagccacagatgggtcttatccttgcgagactcccctgcctactagt

SEQ ID NO: 16 - DNA, 86 bp, synthetic construct
  misc_feature 1..86: note = Synthetic
  source 1..86: mol_type = other DNA
ctcgagtgagcgaggagtctcgcaaggataagaactgtaaagccacagatgggttcttatccttgcgagactccctgcctactagt

SEQ ID NO: 17 - DNA, 86 bp, synthetic construct
  misc_feature 1..86: note = Synthetic
  source 1..86: mol_type = other DNA
ctcgagtgagcgagagtctcgcaaggataagaaactgtaaagccacagatgggtttcttatccttgcgagactcctgcctactagt

SEQ ID NO: 18 - DNA, 86 bp, synthetic construct
  misc_feature 1..86: note = Synthetic
  source 1..86: mol_type = other DNA
ctcgagtgagcgatccttaaatgtttaataaacactgtaaagccacagatgggtgtttattaaacatttaaggactgcctactagt

SEQ ID NO: 19 - RNA, 86 nt, synthetic construct
  misc_feature 1..86: note = Synthetic
  source 1..86: mol_type = other RNA
ctcgagtgagcgcgccatttcgatcttccactatctgtaaagccacagatgggatagtggaagatcgaaatggcatgcctactagt

SEQ ID NO: 20 - RNA, 86 nt, synthetic construct
  misc_feature 1..86: note = Synthetic
  source 1..86: mol_type = other RNA
ctcgagtgagcgaccatttcgatcttccactatgctgtaaagccacagatgggcatagtggaagatcgaaatggctgcctactagt

SEQ ID NO: 21 - RNA, 86 nt, synthetic construct
  misc_feature 1..86: note = Synthetic
  source 1..86: mol_type = other RNA
ctcgagtgagcgccgctggaaggatggctccattctgtaaagccacagatgggaatggagccatccttccagcgatgcctactagt

SEQ ID NO: 22 - RNA, 85 nt, synthetic construct
  misc_feature 1..85: note = Synthetic
  source 1..85: mol_type = other RNA
ctcgagtgagcgagctggaaggatggctccttgctgtaaagccacagatgggcaatggagccatccttccagcgtgcctactagt

SEQ ID NO: 23 - RNA, 86 nt, synthetic construct
  misc_feature 1..86: note = Synthetic
  source 1..86: mol_type = other RNA
ctcgagtgagcgactggaaggatggctccattgtctgtaaagccacagatgggacaatggagccatccttccagctgcctactagt

SEQ ID NO: 24 - RNA, 86 nt, synthetic construct
  misc_feature 1..86: note = Synthetic
  source 1..86: mol_type = other RNA
ctcgagtgagcgatgggggagtctcgcaaggatactgtaaagccacagatgggtatccttgcgagactcccccagtgcctactagt

SEQ ID NO: 25 - RNA, 86 nt, synthetic construct
  misc_feature 1..86: note = Synthetic
  source 1..86: mol_type = other RNA
ctcgagtgagcgcgggggagtctcgcaaggataactgtaaagccacagatgggttatccttgcgagactcccccatgcctactagt

SEQ ID NO: 26 - RNA, 86 nt, synthetic construct
  misc_feature 1..86: note = Synthetic
  source 1..86: mol_type = other RNA
ctcgagtgagcgaggggagtctcgcaaggataagctgtaaagccacagatgggcttatccttgcgagactccccctgcctactagt

SEQ ID NO: 27 - RNA, 86 nt, synthetic construct
  misc_feature 1..86: note = Synthetic
  source 1..86: mol_type = other RNA
ctcgagtgagcgagggagtctcgcaaggataagactgtaaagccacagatgggtcttatccttgcgagactcccctgcctactagt

SEQ ID NO: 28 - RNA, 86 nt, synthetic construct; identical to SEQ ID NO: 27
  misc_feature 1..86: note = Synthetic
  source 1..86: mol_type = other RNA

SEQ ID NO: 29 - RNA, 86 nt, synthetic construct
  misc_feature 1..86: note = Synthetic
  source 1..86: mol_type = other RNA
ctcgagtgagcgagagtctcgcaaggataagaaactgtaaagccacagatgggtttcttatccttgcgagactcctgcctactagt

SEQ ID NO: 30 - RNA, 86 nt, synthetic construct
  misc_feature 1..86: note = Synthetic
  source 1..86: mol_type = other RNA
ctcgagtgagcgatccttaaatgtttaataaacactgtaaagccacagatgggtgtttattaaacatttaaggactgcctactagt

SEQ ID NO: 31 - RNA, 22 nt, synthetic construct
  misc_feature 1..22: note = Synthetic
  source 1..22: mol_type = other RNA
atagtggaagatcgaaatggca

SEQ ID NO: 32 - RNA, 22 nt, synthetic construct
  misc_feature 1..22: note = Synthetic
  source 1..22: mol_type = other RNA
catagtggaagatcgaaatggc

SEQ ID NO: 33 - RNA, 22 nt, synthetic construct
  misc_feature 1..22: note = Synthetic
  source 1..22: mol_type = other RNA
aatggagccatccttccagcga

SEQ ID NO: 34 - RNA, 22 nt, synthetic construct
  misc_feature 1..22: note = Synthetic
  source 1..22: mol_type = other RNA
caatggagccatccttccagcg

SEQ ID NO: 35 - RNA, 22 nt, synthetic construct
  misc_feature 1..22: note = Synthetic
  source 1..22: mol_type = other RNA
acaatggagccatccttccagc

SEQ ID NO: 36 - RNA, 22 nt, synthetic construct
  misc_feature 1..22: note = Synthetic
  source 1..22: mol_type = other RNA
tatccttgcgagactcccccag

SEQ ID NO: 37 - RNA, 22 nt, synthetic construct
  misc_feature 1..22: note = Synthetic
  source 1..22: mol_type = other RNA
ttatccttgcgagactccccca

SEQ ID NO: 38 - RNA, 22 nt, synthetic construct
  misc_feature 1..22: note = Synthetic
  source 1..22: mol_type = other RNA
cttatccttgcgagactccccc

SEQ ID NO: 39 - RNA, 22 nt, synthetic construct
  misc_feature 1..22: note = Synthetic
  source 1..22: mol_type = other RNA
tcttatccttgcgagactcccc

SEQ ID NO: 40 - RNA, 22 nt, synthetic construct
  misc_feature 1..22: note = Synthetic
  source 1..22: mol_type = other RNA
ttcttatccttgcgagactccc

SEQ ID NO: 41 - RNA, 22 nt, synthetic construct
  misc_feature 1..22: note = Synthetic
  source 1..22: mol_type = other RNA
tttcttatccttgcgagactcc

SEQ ID NO: 42 - RNA, 22 nt, synthetic construct
  misc_feature 1..22: note = Synthetic
  source 1..22: mol_type = other RNA
tgtttattaaacatttaaggac

SEQ ID NO: 43 - DNA, 22 bp, synthetic construct
  misc_feature 1..22: note = Synthetic
  source 1..22: mol_type = other DNA
tgccatttcgatcttccactat

SEQ ID NO: 44 - DNA, 22 bp, synthetic construct
  misc_feature 1..22: note = Synthetic
  source 1..22: mol_type = other DNA
gccatttcgatcttccactatg

SEQ ID NO: 45 - DNA, 22 bp, synthetic construct
  misc_feature 1..22: note = Synthetic
  source 1..22: mol_type = other DNA
tcgctggaaggatggctccatt

SEQ ID NO: 46 - DNA, 22 bp, synthetic construct
  misc_feature 1..22: note = Synthetic
  source 1..22: mol_type = other DNA
cgctggaaggatggctccattg

SEQ ID NO: 47 - DNA, 22 bp, synthetic construct
  misc_feature 1..22: note = Synthetic
  source 1..22: mol_type = other DNA
gctggaaggatggctccattgt

SEQ ID NO: 48 - DNA, 22 bp, synthetic construct
  misc_feature 1..22: note = Synthetic
  source 1..22: mol_type = other DNA
ctgggggagtctcgcaaggata

SEQ ID NO: 49 - DNA, 22 bp, synthetic construct
  misc_feature 1..22: note = Synthetic
  source 1..22: mol_type = other DNA
tgggggagtctcgcaaggataa

SEQ ID NO: 50 - DNA, 22 bp, synthetic construct
  misc_feature 1..22: note = Synthetic
  source 1..22: mol_type = other DNA
gggggagtctcgcaaggataag

SEQ ID NO: 51 - DNA, 22 bp, synthetic construct
  misc_feature 1..22: note = Synthetic
  source 1..22: mol_type = other DNA
ggggagtctcgcaaggataaga

SEQ ID NO: 52 - DNA, 22 bp, synthetic construct
  misc_feature 1..22: note = Synthetic
  source 1..22: mol_type = other DNA
gggagtctcgcaaggataagaa

SEQ ID NO: 53 - DNA, 22 bp, synthetic construct
  misc_feature 1..22: note = Synthetic
  source 1..22: mol_type = other DNA
ggagtctcgcaaggataagaaa

SEQ ID NO: 54 - DNA, 22 bp, synthetic construct
  misc_feature 1..22: note = Synthetic
  source 1..22: mol_type = other DNA
gtccttaaatgtttaataaaca

SEQ ID NO: 55 - DNA, 57 bp, synthetic construct
  misc_feature 1..57: note = Synthetic
  source 1..57: mol_type = other DNA
aaaactcgagtgagcgcgccatttcgatcttccactatctgtaaagccacagatggg

SEQ ID NO: 56 - DNA, 56 bp, synthetic construct
  misc_feature 1..56: note = Synthetic
  source 1..56: mol_type = other DNA
ttttactagtaggcatgccatttcgatcttccactatcccatctgtggctttacag

SEQ ID NO: 57 - DNA, 57 bp, synthetic construct
  misc_feature 1..57: note = Synthetic
  source 1..57: mol_type = other DNA
aaaactcgagtgagcgaccatttcgatcttccactatgctgtaaagccacagatggg

SEQ ID NO: 58 - DNA, 56 bp, synthetic construct
  misc_feature 1..56: note = Synthetic
  source 1..56: mol_type = other DNA
ttttactagtaggcagccatttcgatcttccactatgcccatctgtggctttacag

SEQ ID NO: 59 - DNA, 57 bp, synthetic construct
  misc_feature 1..57: note = Synthetic
  source 1..57: mol_type = other DNA
aaaactcgagtgagcgccgctggaaggatggctccattctgtaaagccacagatggg

SEQ ID NO: 60 - DNA, 56 bp, synthetic construct
  misc_feature 1..56: note = Synthetic
  source 1..56: mol_type = other DNA
ttttactagtaggcatcgctggaaggatggctccattcccatctgtggctttacag

SEQ ID NO: 61 - DNA, 57 bp, synthetic construct
  misc_feature 1..57: note = Synthetic
  source 1..57: mol_type = other DNA
aaaactcgagtgagcgagctggaaggatggctccattgctgtaaagccacagatggg

SEQ ID NO: 62 - DNA, 56 bp, synthetic construct
  misc_feature 1..56: note = Synthetic
  source 1..56: mol_type = other DNA
ttttactagtaggcacgctggaaggatggctccattgcccatctgtggctttacag

SEQ ID NO: 63 - DNA, 57 bp, synthetic construct
  misc_feature 1..57: note = Synthetic
  source 1..57: mol_type = other DNA
aaaactcgagtgagcgactggaaggatggctccattgtctgtaaagccacagatggg

SEQ ID NO: 64 - DNA, 56 bp, synthetic construct
  misc_feature 1..56: note = Synthetic
  source 1..56: mol_type = other DNA
ttttactagtaggcagctggaaggatggctccattgtcccatctgtggctttacag

SEQ ID NO: 65 - DNA, 57 bp, synthetic construct
  misc_feature 1..57: note = Synthetic
  source 1..57: mol_type = other DNA
aaaactcgagtgagcgatgggggagtctcgcaaggatactgtaaagccacagatggg

SEQ ID NO: 66 - DNA, 56 bp, synthetic construct
  misc_feature 1..56: note = Synthetic
  source 1..56: mol_type = other DNA
ttttactagtaggcactgggggagtctcgcaaggatacccatctgtggctttacag

SEQ ID NO: 67 - DNA, 57 bp, synthetic construct
  misc_feature 1..57: note = Synthetic
  source 1..57: mol_type = other DNA
aaaactcgagtgagcgcgggggagtctcgcaaggataactgtaaagccacagatggg

SEQ ID NO: 68 - DNA, 56 bp, synthetic construct
  misc_feature 1..56: note = Synthetic
  source 1..56: mol_type = other DNA
ttttactagtaggcatgggggagtctcgcaaggataacccatctgtggctttacag

SEQ ID NO: 69 - DNA, 57 bp, synthetic construct
  misc_feature 1..57: note = Synthetic
  source 1..57: mol_type = other DNA
aaaactcgagtgagcgaggggagtctcgcaaggataagctgtaaagccacagatggg

SEQ ID NO: 70 - DNA, 56 bp, synthetic construct
  misc_feature 1..56: note = Synthetic
  source 1..56: mol_type = other DNA
ttttactagtaggcagggggagtctcgcaaggataagcccatctgtggctttacag

SEQ ID NO: 71 - DNA, 57 bp, synthetic construct
  misc_feature 1..57: note = Synthetic
  source 1..57: mol_type = other DNA
aaaactcgagtgagcgagggagtctcgcaaggataagactgtaaagccacagatggg

SEQ ID NO: 72 - DNA, 56 bp, synthetic construct
  misc_feature 1..56: note = Synthetic
  source 1..56: mol_type = other DNA
ttttactagtaggcaggggagtctcgcaaggataagacccatctgtggctttacag

SEQ ID NO: 73 - DNA, 57 bp, synthetic construct
  misc_feature 1..57: note = Synthetic
  source 1..57: mol_type = other DNA
aaaactcgagtgagcgaggagtctcgcaaggataagaactgtaaagccacagatggg

SEQ ID NO: 74 - DNA, 56 bp, synthetic construct
  misc_feature 1..56: note = Synthetic
  source 1..56: mol_type = other DNA
ttttactagtaggcagggagtctcgcaaggataagaacccatctgtggctttacag

SEQ ID NO: 75 - DNA, 57 bp, synthetic construct
  misc_feature 1..57: note = Synthetic
  source 1..57: mol_type = other DNA
aaaactcgagtgagcgagagtctcgcaaggataagaaactgtaaagccacagatggg

SEQ ID NO: 76 - DNA, 56 bp, synthetic construct
  misc_feature 1..56: note = Synthetic
  source 1..56: mol_type = other DNA
ttttactagtaggcaggagtctcgcaaggataagaaacccatctgtggctttacag

SEQ ID NO: 77 - DNA, 57 bp, synthetic construct
  misc_feature 1..57: note = Synthetic
  source 1..57: mol_type = other DNA
aaaactcgagtgagcgatccttaaatgtttaataaacactgtaaagccacagatggg

SEQ ID NO: 78 - DNA, 56 bp, synthetic construct
  misc_feature 1..56: note = Synthetic
  source 1..56: mol_type = other DNA
ttttactagtaggcagtccttaaatgtttaataaacacccatctgtggctttacag

SEQ ID NO: 79-82 - skipped (4 entries)

SEQ ID NO: 83 - DNA, 747 bp, Mus musculus
  source 1..747: mol_type = other DNA
atggctcccggggctccctcctccagccccagccctatcctggctgccctgctcttctcttctttggtgctctctccagccctggccattgtggtttacacggacagggaaatctatggtgccgtgggctcccaggtgaccctgcactgctccttctggtccagtgaatgggtctcagatgacatctcttttacctggcgctaccagcctgaagggggccgagatgccatttcgatcttccactatgccaagggacaaccttacatcgatgaggtggggaccttcaaagagcgcatccagtgggtaggggaccctcgctggaaggatggctccattgtcatacacaacctagactacagtgacaacggcactttcacatgtgatgtcaaaaacccaccggacatagtgggcaagacctctcaggtcacgctctatgtctttgaaaaagtgcccactaggtatggggtggtgttgggagcagtgatcgggggcatcctcggggtggtgctgttgctgctgttgctcttctacctgattcggtactgctggctgcgcaggcaggctgccctgcagagaaggctcagtgccatggagaaggggagatttcacaaatcttcgaaggactcctcgaagcgagggcggcagacgccagtgctgtatgccatgctggaccacagccgaagcaccaaagctgccagtgagaagaaatcaaaagggctgggggagtctcgcaaggataagaaatag

SEQ ID NO: 84 - DNA, 1087 bp, Mus musculus
  source 1..1087: mol_type = other DNA
cggttagcgggccgggcggggggtcgggggtctgcgacggagtcctccaaaggctctcaggtggtggtcatcgagatggagcttcgcaaagatgagcagagctcggagctccggcctgcagtcaagtcccccagtagaaccagcctcaagaacgccctcaagaacatgatgggcctggactcggacaagtgaccgtcacatccaggccctgtcagagcaggggacctaggctcctctttttcccgggtctaggtgctttcctcttgctcctcagccctgccctgcccttacctcccaatgagatgtaaagtttcattccaaccttcctttccaagtcttgtactcctccttcgcctttacccctcggctttccaggagcctagggactaaccctactcctcgcctgccccaagggctgtgtgtttggtgcctgtctctcagacaccgagaagaaagggaccttgatagcctctgcccaactccaggccacttggccttcccatctctcccccccccgcccccccccccagctgatcccctggctccttcctgctctccctttcctaccttctggtggcccagctccacactctgtcctcgggctcaaatccacatcgtgcagagcagactctccatcagggtttatttaggttgctgagtattttttatttgcccatccattctttcgtttgtttacctgagcccacggccccatgaccgaggaccaatgacgtcatgtggcttttgcagttccccactaagtctttactggagagccaggcaggcaagcagcgcataatcgatgccctctgaccactacggagcacagttctgggactccgagatttggggaaggaaagaaaaaaaaatggatcctcacaggtcaggggtgcagtagggggctttaagaaatgtcttcaaaataaataatcagaacaggaaacgccagcgagaatgggaaggaaaggaagtggctacacccccgggcacagtggactgttaggacttttctacattctgtatatttctcctaccatctccaaatgtccttaaatgtttaataaacactgacatttccagaagctgc